(19) United States
(12) Patent Application Publication
(10) Pub. No.: US 2026/0053931 A1
(43) Pub. Date: Feb. 26, 2026

(54) MDM2 TARGETING PROTACS

(71) Applicants: THE WISTAR INSTITUTE OF ANATOMY AND BIOLOGY, Philadelphia, PA (US); THOMAS JEFFERSON UNIVERSITY, Philadelphia, PA (US)

(72) Inventors: Joseph M. SALVINO, Philadelphia, PA (US); Christine M. EISCHEN, Chester Springs, PA (US)

(73) Assignees: THE WISTAR INSTITUTE OF ANATOMY AND BIOLOGY, Philadelphia, PA (US); THOMAS JEFFERSON UNIVERSITY, Philadelphia, PA (US)

(21) Appl. No.: 19/125,010

(22) PCT Filed: Jan. 5, 2024
(86) PCT No.: PCT/US2024/010472

§ 371 Date: Apr. 28, 2025

Related U.S. Application Data
(60) Provisional application No. 63/478,667, filed on Jan. 5, 2023.

(51) Int. Cl. A61K 47/54; A61K 31/496; A61P 35/00

(52) U.S. Cl. CPC A61K 47/545; A61K 31/496; A61P 35/00

(57) ABSTRACT

Disclosed herein are compounds of the formulas: (I) as well as analogs thereof, wherein the variables are defined herein. Also provided are pharmaceutical compositions of these compounds. In some aspects, the compounds and compositions provided herein may be used to degrade Mdm2. Also provided are methods of administering compounds and compostions provided herein to a patient in need thereof, for example, for the treatment of cancers.

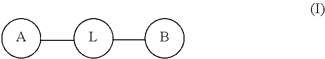

This application is U.S. National Phase Application under 35 U.S.C. § 371 of International Patent Application No. PCT/US2024/010472, filed Jan. 5, 2024, which claims the benefit of priority to U.S. Provisional Application No. 63/478,667, filed on Jan. 5, 2023, the entire contents of which are hereby incorporated by reference.

This invention was made with government support under CA180262, CA272645 and DE033025 awarded by the National Institutes of Health and W81XWH-21-1-0303 and W81XWH-19-1-0212 awarded by the United States Department of Defense. The government has certain rights in the invention.

### INCORPORATION OF SEQUENCE LISTING

The sequence listing that is contained in the file named “WISTP0015US.xml”, which is 2,802 bytes (as measured in Microsoft Windows) and was created on Oct. 10, 2025, is filed herewith by electronic submission and is incorporated by reference herein.

### BACKGROUND

### I. Field

This disclosure relates to the fields of biology, pharmacology, medicine, and chemistry.

In particular, new compounds, compositions, and methods of treatment related to the treatment of cancers are disclosed.

### II. Description of Related Art

The tumor suppressor p53, a transcription factor, has an essential role in the prevention of human cancer (Levine, 2020). In the absence of cellular stress, the p53 protein is maintained at low levels due to binding to Mdm2, an E3 ubiquitin ligase (Eischen & Lozano, 2014). Mdm2's E3 ligase activity polyubiquitinates p53 resulting in its degradation by the 26S proteasome. p53 is activated by a variety of tumorigenic stresses, which include aberrant oncogene signaling and DNA damage. These stresses activate p53 causing an increase in its transcriptional activity, resulting cell cycle arrest, cellular senescence, and/or apoptosis (Levine, 2020).

The most well-known function of Mdm2 is as a negative regulator of p53 actions through an auto-regulatory negative feedback loop in which increased p53 levels activate its transcription of Mdm2. Mdm2 then binds p53 blocking its transactivation domain and targets it for proteasomal degradation (Eischen & Lozano, 2014). Since Mdm2 is overproduced in many human cancers (Oliner et al., 2016; Rayburn et al., 2005), targeting its activity is a major focus of drug discovery efforts. Drugs targeting Mdm2, such as RG7112, Idasanutlin, and AMG232 have demonstrated clinical efficacy, and Idasanutlin has progressed into Phase 3 clinical trials (Skalniak et al., 2018; Burgess et al., 2016). However, these drugs have significant undesired toxicities associated with increased levels of Mdm2. Mdm2 inhibitors bind and stabilize the Mdm2 protein by specific binding to the hydrophobic binding pocket of Mdm2. The combination of stabilization of Mdm2 by inhibitor binding and increased p53 levels, which activate Mdm2 transcription, results in significantly increased levels of Mdm2 (Hunziker et al., 2010; Harris & Levine, 2005). Higher Mdm2 levels engage both p53-dependent and p53-independent functions of Mdm2 (Riley & Lozano, 2012; Eischen & Lozano, 2014; Bouska & Eischen 2009), leading to toxicities (thrombocytopenia and neutropenia), which limits prolonged drug treatment in patients treated with Mdm2 inhibitors (Ray-Coquard et al., 2012). In addition, prolonged treatment with Mdm2 inhibitors leads to resistance caused by p53 mutation (Skalniak et al., 2018; Aziz et al., 2011; Michaelis et al., 2011; Jung et al., 2016).

Half of all human cancers have inactivated p53 by mutation or deletion (Olivier et al., 2010). To test the requirements of Mdm2 in the absence of p53, Mdm2 was inducibly deleted in primary murine p53-null lymphoma and sarcoma cells. Mdm2 loss resulted in apoptosis in vitro and in vivo in p53-null cancers, with a significantly reduced tumor burden and survival benefit (Feeley et al., 2017). This suggests that Mdm2 may be required for the survival of cancer cells that lack p53. It has been reported that Mdm2 has p53-independent functions by binding and regulating other proteins, such as p53 family members, p73 and p63, Nbs1 in the Mrel 1-Rad50-Nbs1 DNA break repair complex and other proteins that impact cancer (Riley & Lozano, 2012; Melo & Eischen, 2012; Ongkeko et al., 1999; Zeng et al., 1999; Wu & Leng, 2015). Notably, it was shown that p73 was induced and necessary for mediating the apoptotic effects of Mdm2 loss in the absence of p53 in murine cancer cells (Feeley et al., 2017). The evaluation of human cancer cells that harbor mutant p53 also show that Mdm2 loss induces p73 and apoptosis, indicating this is not a mouse-specific effect. Importantly, p73 has a homologous N-terminal transactivation domain to p53 that binds to Mdm2 in the same site as p53, shown by biophysical studies (Shin et al., 2015), suggesting a remarkably close mimicry of the Mdm2 recognition mechanism among p53 family members. Additionally, unlike p53, p73 is rarely inactivated in human cancers (Dotsch et al., 2010; Venkatanarayan et al., 2016).

Therefore, there remains a need to develop Mdm2 targeting molecules without the side effects of the known Mdm2 inhibitors and to be able to target p53-inactivated cancers.

### SUMMARY

In some aspects, the present disclosure provides compounds, pharmaceutical compositions, and methods for their use in the treatment of cancers. In some embodiments, the present disclosure relates to Mdm2 degrading molecules such as an Mdm2 targeting PROTAC. In particular, the Mdm2 degrading molecules are described that are able to target both wild-type, mutated, inactivated, and null p53 cancers.

In a further aspect, the present disclosure provides compounds of the formula:

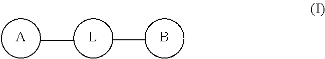

wherein:
   A is a mdm2 inhibitor;
   B is a ubiquitin ligase ligand; and
   L is a linker group, wherein the linker group forms a covalent bond between the linker group and the mdm2 inhibitor and a covalent bond between the ubiquitin ligase ligand;
or a pharmaceutically acceptable salt thereof.

In some embodiments, the mdm2 inhibitor is further defined by the formula:

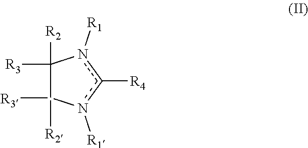

wherein:
   R₁ and R₁′ are each independently a group of the formula:

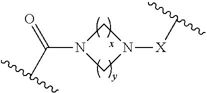

wherein:
   X is -alkanediyl_{(C≤8)}—C(O)— or substituted -alkanediyl_{(C≤8)}—C(O)—; and
   x or y are each independently 1, 2, or 3;
provided that either R₁ or R₁′ is absent when the nitrogen to which it is attached is a part of a double bond;
R₂ and R₂′ are each independently hydrogen or alkyl_{(C≤8)}, cycloalkyl_{(C≤8)}, alkenyl_{(C≤8)}, alkynyl_{(C≤8)}, aryl_{(C≤12)}, aralkyl_{(C≤12)}, or a substituted version of any of these groups;
R₃ and R₃′ are each independently alkyl_{(C≤8)}, cycloalkyl_{(C≤8)}, alkenyl_{(C≤8)}, alkynyl_{(C≤8)}, aryl_{(C≤12)}, heteroaryl_{(C≤12)}, aralkyl_{(C≤12)}, or a substituted version of any of these groups; and
R₄ is cycloalkyl_{(C≤8)}, aryl_{(C≤8)}, heteroaryl_{(C≤8)}, heterocycloalkyl_{(C≤8)}, or a substituted version thereof, wherein the group may be further substituted by one or more alkyl_{(C≤8)}, alkoxy_{(C≤8)}, alkylamino_{(C≤8)}, dialkylamino_{(C≤8)}, or a substituted version of these groups;
or a pharmaceutically acceptable salt thereof.

In some embodiments, the mdm2 inhibitor is further defined:

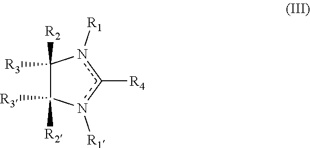

wherein:
   R₁ and R₁′ are each independently a group of the formula:

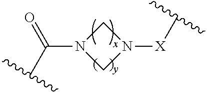

wherein:
   X is -alkanediyl_{(C≤8)}—C(O)— or substituted -alkanediyl_{(C≤8)}—C(O)—; and
   x or y are each independently 1, 2, or 3;
provided that either R₁ or R₁′ is absent when the nitrogen to which it is attached is a part of a double bond;
R₂ and R₂′ are each independently hydrogen or alkyl_{(C≤8)}, cycloalkyl_{(C≤8)}, alkenyl_{(C≤8)}, alkynyl_{(C≤8)}, aryl_{(C≤12)}, aralkyl_{(C≤12)}, or a substituted version of any of these groups;
R₃ and R₃′ are each independently alkyl_{(C≤8)}, cycloalkyl_{(C≤8)}, alkenyl_{(C≤8)}, alkynyl_{(C≤8)}, aryl_{(C≤12)}, heteroaryl_{(C≤12)}, aralkyl_{(C≤12)}, or a substituted version of any of these groups; and
R₄ is cycloalkyl_{(C≤8)}, aryl_{(C≤8)}, heteroaryl_{(C≤8)}, heterocycloalkyl_{(C≤8)}, or a substituted version thereof, wherein the group may be further substituted by one or more alkyl_{(C≤8)}, alkoxy_{(C≤8)}, alkylamino_{(C≤8)}, dialkylamino_{(C≤8)}, or a substituted version of these groups;
or a pharmaceutically acceptable salt thereof.

In some embodiments, the compounds are further comprising:

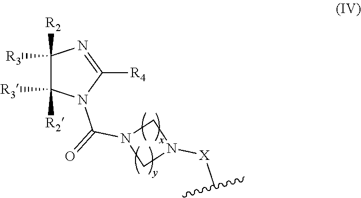

wherein:
   R₂ and R₂′ are each independently hydrogen or alkyl_{(C≤8)}, cycloalkyl_{(C≤8)}, alkenyl_{(C≤8)}, alkynyl_{(C≤8)}, aryl_{(C≤12)}, aralkyl_{(C≤12)}, or a substituted version of any of these groups;
   R₃ and R₃′ are each independently alkyl_{(C≤8)}, cycloalkyl_{(C≤8)}, alkenyl_{(C≤8)}, alkynyl_{(C≤8)}, aryl_{(C≤12)}, heteroaryl_{(C≤12)}, aralkyl_{(C≤12)}, or a substituted version of any of these groups; and
   R₄ is cycloalkyl_{(C≤8)}, aryl_{(C≤8)}, heteroaryl_{(C≤8)}, heterocycloalkyl_{(C≤8)}, or a substituted version thereof, wherein the group may be further substituted by one or more alkyl_{(C≤8)}, alkoxy_{(C≤8)}, alkylamino_{(C≤8)}, dialkylamino_{(C≤8)}, or a substituted version of these groups;
or a pharmaceutically acceptable salt thereof.

In some embodiments, R₁ is

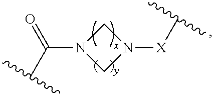

wherein: X is -alkanediyl_{(C≤8)}—C(O)— or substituted -alkanediyl_{(C≤8)}—C(O)—; and x or y are each independently 1, 2, or 3. In some embodiments, X is -alkanediyl_{(C≤8)}—C(O)—. In some embodiments, the alkanediyl_{(C≤8)} is methylene. In some embodiments, x is 1 or 2. In some embodiments, x is 2. In some embodiments, y is 1 or 2. In some embodiments, y is 2.

In some embodiments, R₂ is alkyl_{(C≤8)} or substituted alkyl_{(C≤8)}. In some embodiments, R₂ is alkyl_{(C≤8)} such as methyl. In some embodiments, R₂′ is alkyl_{(C≤8)} or substituted alkyl_{(C≤8)}. In some embodiments, R₂′ is alkyl_{(C≤8)} such as methyl.

In some embodiments, R₃ is aryl_{(C≤12)} or substituted aryl_{(C≤12)}. In some embodiments, R₃ is substituted aryl_{(C≤12)} such as chlorophenyl. In some embodiments, R₃ is 4-chlorophenyl. In some embodiments, R₃′ is aryl_{(C≤12)} or substituted aryl_{(C≤12)}. In some embodiments, R₃′ is substituted aryl_{(C≤12)} such as chlorophenyl. In some embodiments, R₃′ is 4-chlorophenyl.

In some embodiments, R₄ is aryl_{(C≤8)} or substituted aryl_{(C≤8)}, wherein the group is further substituted with one or more alkyl_{(C≤8)}, alkoxy_{(C≤8)}, alkylamino_{(C≤8)}, dialkylamino_{(C≤8)}, or a substituted version of these groups. In some embodiments, R₄ is an aryl_{(C≤8)}, which is further substituted with one or more alkyl_{(C≤8)}, alkoxy_{(C≤8)}, or a substituted version of either group. In some embodiments, the aryl is further substituted with one or more alkyl_{(C≤8)} or substituted alkyl_{(C≤8)}. In some embodiments, the aryl is further substituted with one or more alkyl_{(C≤8)}. In some embodiments, the aryl is further substituted with a t-butyl group. In other embodiments, the aryl is further substituted with one or more alkoxy_{(C≤8)} or substituted alkoxy_{(C≤8)}. In some embodiments, the aryl is further substituted with one or more alkoxy_{(C≤8)}. In some embodiments, the aryl is further substituted with an ethoxy group. In some embodiments, the aryl is further substituted with a t-butyl group and an ethoxy group. In some embodiments, R₄ is 2-ethoxy-4-t-butylphenyl. In some embodiments, the mdm2 inhibitor is further defined as:

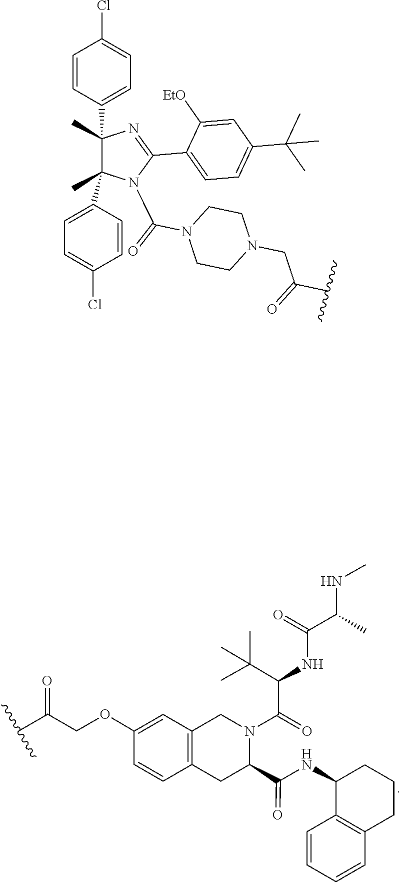 [SMILES: *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1 ; *C(=O)COc1ccc2c(c1)CN(C(=O)[C@H](NC(=O)[C@@H](C)NC)C(C)(C)C)[C@@H](C(=O)N[C@H]1CCCc3ccccc31)C2]

In some embodiments, B is a Cereblon (CRBN) ligand, a Von-Hippel Lindau (VHL) ligand, a mouse double minute 2 homolog (MDM2) ligand, or a cellular inhibitor of apoptosis protein 1 (cIAP1) ligand. In some embodiments, B is a CRBN ligand. In some embodiments, B is a group of the formula:

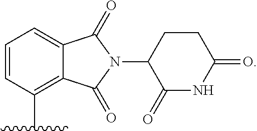 [SMILES: *c1cccc2c1C(=O)N(C1CCC(=O)NC1=O)C2=O]

In other embodiments, B is a VHL ligand. In some embodiments, B is a group of the formula:

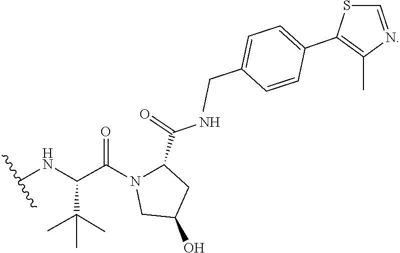

In other embodiments, B is a MDM2 ligand. In some embodiments, B is a group of the formula:

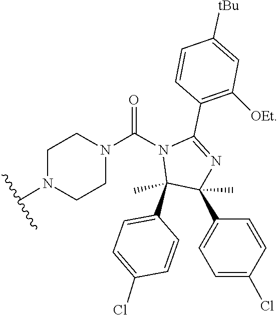 [SMILES: *N1CCN(C(=O)N2C(c3ccc(C(C)(C)C)cc3OCC)=N[C@@](C)(c3ccc(Cl)cc3)[C@@]2(C)c2ccc(Cl)cc2)CC1]

In other embodiments, B is a cIAP ligand. In some embodiments, B is a group of the formula:

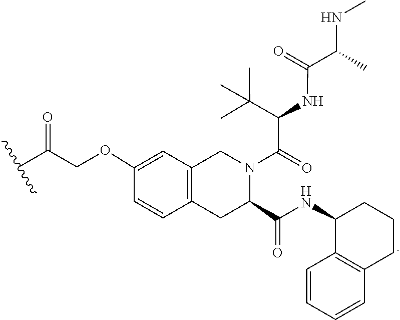 [SMILES: *C(=O)COc1ccc2c(c1)CN(C(=O)[C@H](NC(=O)[C@@H](C)NC)C(C)(C)C)[C@@H](C(=O)N[C@H]1CCCc3ccccc31)C2]

In some embodiments, L is a covalent bond. In other embodiments, L is a linker group of the formula:

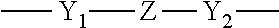

wherein:
   Y₁ and Y₂ are each independently selected from —O—, —NRₐ—, —C(O)—, —C(O)O—, —OC(O)—, —C(O)NRₐ—, —NRₐC(O)—, —S(O)ₐ—, —S(O)ₐO—, —OS(O)ₐ—, —OS(O)ₐO—, wherein: Rₐ is hydrogen, alkyl(c>6), or substituted alkyl(c>6); and a is 0, 1, or 2; and
   Z is alkanediyl_{(C≤12)}, one or more amino acid residues, or a group of the formula: —((Z₁)O)_{z}—, wherein: Z₁ is an alkanediyl_{(C1-4)} or a substituted alkanediyl_{(C1-4)} and z is 1-10 repeating units; or a combination thereof.

In some embodiments, Y₁ is —NRₐ—. In some embodiments, Rₐ is hydrogen. In other embodiments, Y₁ is —C(O)—. In some embodiments, Y₂ is —NRₐ—. In some embodiments, Rₐ is hydrogen. In other embodiments, Y₂ is —C(O)—. In some embodiments, Z is alkanediyl_{(C≤12)} or a substituted alkanediyl_{(C≤12)}. In some embodiments, Z is alkanediyl_{(C≤12)} such as ethanediyl, propanediyl, or butanediyl. In other embodiments, Z is —((Z₁)O)_{z}—. In some embodiments, Z₁ is alkanediyl(ci-4). In some embodiments, Z₁ is ethanediyl or 2-methylpropane-1,2-diyl. In some embodiments, Z₁ is ethanediyl. In some embodiments, z is 1, 2, 3, or 4. In some embodiments, z is 1. In other embodiments, z is 2. In other embodiments, z is 3. In some embodiments, Z is a combination of alkanediyl(cn₂) or a substituted alkanediyl_{(C≤12)} and —((Z₁)O)_{z}—.

In some embodiments, the compound is further defined as:

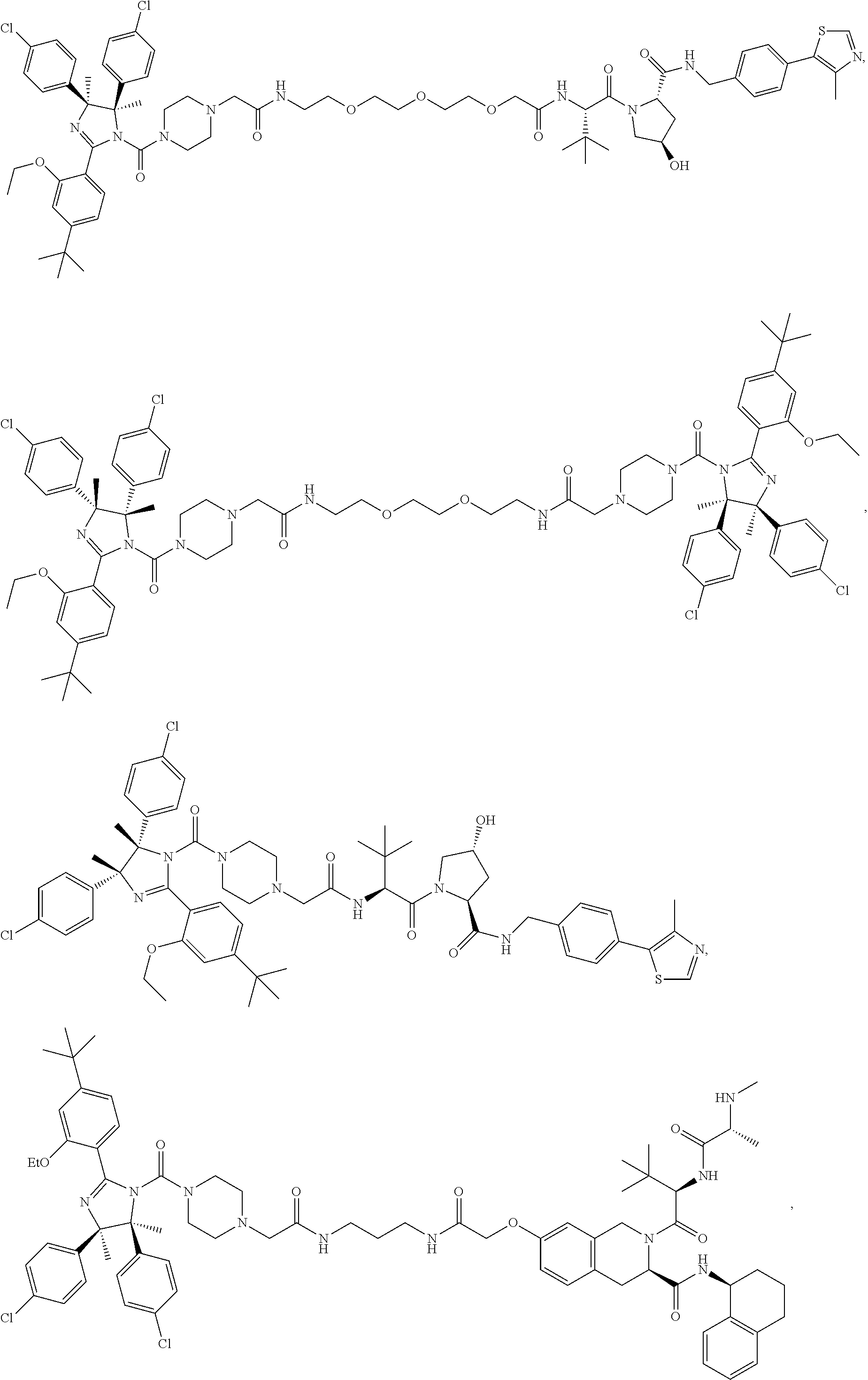

or;
or a pharmaceutically acceptable salt thereof.

In some embodiments, the compound is further defined as:

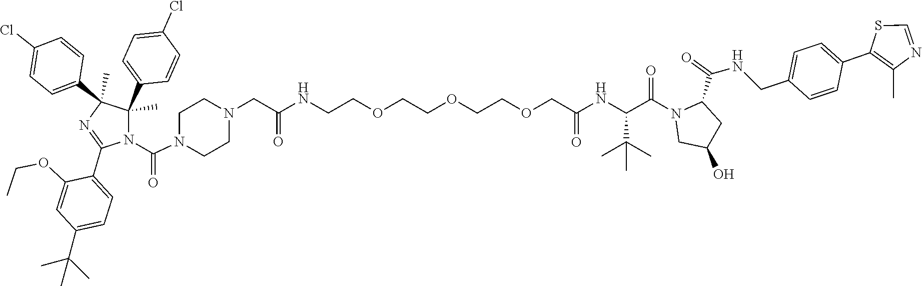 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCOCCOCCOCC(=O)N[C@H](C(=O)N2C[C@H](O)C[C@H]2C(=O)NCc2ccc(-c3scnc3C)cc2)C(C)(C)C)CC1]

or a pharmaceutically acceptable salt thereof.

In still another aspect, the present disclosure provides pharmaceutical composition comprising:
(A) a compound described herein; and
(B) a pharmaceutically acceptable excipient.

In some embodiments, the pharmaceutical compositions are formulated for oral administration or administration via injection. In some embodiments, the pharmaceutical compositions are formulated as a unit dose.

In still another aspect, the present disclosure provides methods of treating a disease or disorder in a patient in need thereof comprising administering to the patient in need thereof a therapeutically effective amount of a compound or composition described herein. In some embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some embodiments, the cancer comprises a mutated p53 gene. In some embodiments, the cancer comprises a deleted p53 gene. In some embodiments, the cancer is breast cancer such as triple negative breast cancer. In other embodiments, the cancer is ovarian cancer. In other embodiments, the cancer is Burkitt lymphoma. In other embodiments, the cancer is diffuse large B cell lymphoma. In some embodiments, the diffuse large B cell lymphoma is subtype ABC. In other embodiments, the diffuse large B cell lymphoma is subtype GCB. In other embodiments, the cancer is a B-cell leukemia such as B-cell acute lymphocytic leukemia. In other embodiments, the cancer is a T-cell leukemia such as T-cell acute lymphocytic leukemia. In some embodiments, the cancer is a lung cancer such as lung adenocarcinoma. In other embodiments, the cancer is head and neck cancer. In other embodiments, the cancer is a sarcoma.

In some embodiments, the methods further comprise administering a second therapy. In some embodiments, the second therapy is surgery, a second chemotherapeutic, radiotherapy, or immunotherapy. In some embodiments, the compound is administered once. In other embodiments, the compound is administered two or more times.

In another aspect, the present disclosure provides methods of inhibiting cell replication by modulating mdm2 comprising contacting the cell with a compound or composition described herein.

In still yet another aspect, the present disclosure provides methods of modulating the activity of mdm2 in a cell comprising contacting the cell with a compound or composition described herein.

In some embodiments, the method is performed in vitro. In some embodiments, the method is performed in vivo. In some embodiments, the method is performed ex vivo.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows YX-02-020 (IK-1-100) as a racemic mixture as shown by chiral chromatography.

FIG. 2 shows that YX-02-023 (IK-1-100) was resolved to each enantio-enriched isomer by chiral chromatography.

FIG. 3 shows the synthesis of enantio-enriched YX-02-030, using IK-01-100 P1 to make IK-01-186. Using IK-01-100 P2 the other enantio-enriched YX-02-030 isomer was synthesized, IK-01-189.

FIG. 4 shows the ¹H NMR spectra of IK-01-186, consistent with the structure.

FIG. 5 shows the ¹H NMR Spectra of IK-01-189, consistent with the structure.

FIG. 6 shows the inhibition of YX-02-023 and YX-02-030 in the HTRF Mdm2 binding assay.

FIG. 7 show the single enriched enantiomers separated by chiral chromatography. Absolute configuration is not known. IK-01-189 is about 90-fold more potent than IK-01-186. IK-01-186 is one enantiomer enriched isomer of YX-02-030. IK-01-189 is the other enantiomer enriched isomer of YX-02-030. JG-02-071 is a scale up re-synthesis of YX-02-030.

FIGS. 8A-8E show the dose response curves and IC₅₀ values of scale batch and single enriched enantiomers of YX-02-030 and binding IC₅₀ values. A. IC₅₀ values and parameters. B-C. Dose response curves for YX-02-030 single enantiomers, IK-01-186 and IK-01-189. D-E. Dose response curves for JG-02-071 scale up re-synthesis of YX-02-030 and original YX-02-030 small scale batch.

FIG. 9 shows the evaluation of the ternary complex formation. YX-02-030 and the single enriched enantiomer of YX-02-03, IK-01-189, efficiently form a ternary complex between Mdm2-VHL and PROTAC as shown in the AlphaScreen assay. The less potent single enriched enantiomer of YX-02-030, IK-01-186, did not form a ternary complex in this assay.

FIGS. 10A-10D show that Mdm2 loss inhibits growth and induces apoptosis of p53-null lymphoma cells. A. Western blot for Mdm2 protein. B-D. Vehicle control (EtOH) or 4-OHT was added to Mdm2^{fl/flp}53^{-/-} lymphomas expressing CreER^{T2} and GFP or GFP alone.

Proliferation (MTS assay, quadruplicate), B. Annexin-V C. sub-G1 DNA D. measured (B-D, triplicates); *p<0.01.

FIGS. 11A-11C show that loss of Mdm2 inhibits lymphoma growth in vivo and prolongs survival of mice. A. Tumor volumes in mice (number indicated) injected (subcutaneously) with CreER expressing Mdm2^{fl/fl}p53-null lymphoma cells and administered tamoxifen (Tam) or vehicle control (corn oil) starting day 15 were measured (*p<0.0006). B.

Kaplan-Meier survival curves of the mice in A. Arrow indicates day tamoxifen or oil administration began. C. Evaluation of tumors harvested 48 hours after Tam or vehicle control administration showed loss of Mdm2, increased Annexin V positivity, and fragmented (sub-G1) DNA.

FIGS. 12A-12C show TNBC cells expressing three independent MDM2 shRNA or non-targeting control shRNA (shNT) and Western blots performed for the proteins indicated (A), MTT growth assays (quadruplicate, 24 hr intervals, B), and apoptosis (C) measured by Annexin-V positivity (triplicate, left) and apoptotic subG1 DNA content (triplicate, right).

Mean±SD (B, C). *P<0.0001, comparing PROTAC to control compounds and DMSO vehicle control. For B and C, *P<0.00051, comparing MDM2 shRNA to non-targeting control shRNA. 2-way ANOVA (B, C (left)), 1-way ANOVA (C (right)), and unpaired 2-tailed 1-test.

FIG. 12D shows Spearman's correlation of differential gene expression of RNA-seq data (triplicates) from MDA-MB-231 and MDA-MB-436 cells treated with MDM2-PROTAC versus expressing MDM2 shRNA, each first compared to their respective DMSO vehicle control or non-targeting shRNA control. Correlation coefficients (p) and P-values indicated.

FIGS. 13A-13D show HTRF IC₅₀ values and SPR data that generally correlates with the Mdm2-p53 HTRF binding assay. A. Sensograms for RG7112 B. Sensograms for PROTAC YX-02-228. C-D. Inhibition of compounds RG7112, Nutlin3a, YX-02-023, YX-02-041, YX-02-228, YX-02-030, AC-02-049, AC-03-026 (Idasanutlin).

FIGS. 14A-14D show the Mdm2 ligands and key binding interactions. A. Mdm2 ligands developed using rational structure-based design. B-D. X-ray structures of RG7112, p53, and p73 in complex with Mdm2.

FIGS. 15A-15C show a series of bi-functional molecules treated and evaluated for protein level in the assay over time. A. Significantly increased Mdm2 levels shown by most compounds. B. Stabilization of p53 in the wt p53 cancer cell line. C. Degradation of reference compound MD-224.

FIGS. 16A-16C show the degradation of Mdm2 at varying concentrations. A-B. The reported Mdm2 degrader, MD-224, did not reduce Mdm2 protein level, showing no effect or slightly increased protein levels. C. Time course of 2 h-48 hr.

FIGS. 17A-17C show the effect of treatment on live cells at varying concentrations. A-B. YX-02-030 and YX-02-228 reduced the number of live mut p53 MDA-MD-231 cells remaining after 24 h and 48 h treatment, while MD-224 showed no cytotoxic effect. C. YX-02-228 treatment over time.

FIGS. 18A-18C show that Mdm2 PROTAC targets Mdm2 and is not functioning as molecular glue. A-B. Murine Mdm2+/+p53-null sarcoma cells treated with Mdm2 PROTAC (YX-02-030), control YX-02-023 (−), or vehicle (DMSO). A. Western blots after 24 hrs (cleaved caspase 3, CC3. Mdm2^{-/-}p53^{-/-} fibroblasts were a control for the Western blot). B. Dose response curve (MTT assay, 48 hrs). C. Dose response curves of murine Mdm2^{-/-}p53^{-/-} sarcoma cells treated with Mdm2 PROTAC (YX-02-030), negative controls (−), a Bcl2/Bclx/Bclw inhibitor (+), or vehicle control (MTT assay, quadruplicate, 48 hrs- MTT assay, B-C graphed relative to DMSO).

FIGS. 19A-19K show that the MDM2-PROTAC (YX-02-030) binds MDM2 with high affinity, recruits VHL, and targets MDM2 for proteosome degradation. A. Chemical structure of MDM2-PROTAC. B-C. MDM2:p53 and MDM2:VHL-HIF1α peptide binding inhibition determined by homogeneous time-resolved fluorescence (HTRF, triplicate, mean±SEM, B) and surface plasmon resonance (SPR, triplicate, mean±SD, C. Binding assays by the indicated compounds. Graphical data is representative of 3 separate experiments. D. AlphaScreen of ternary complex using GST-MDM2 and HIS-VHL; mean±SD. E-F. Western blot analysis for the proteins indicated following a dose-titration (E, 16 hr) and time course (F, 6 μM) with the PROTAC or vehicle control (DMSO, −) in MDA-MB-231 (p53-mutant) and MDA-MB-436 (p53-delete) cells. G-K. Western blot analysis for the proteins indicated following treatment with the MDM2-PROTAC and/or the neddylation activating enzyme inhibitor MLN4924 G.

The proteasome inhibitor MG132. H. RG7112. I. VHL-Amine. J. The VHL small molecule inhibitor VH298. K. DMSO vehicle control (−).

FIGS. 20A-20D show the synthesis and binding analysis of the MDM2-PROTAC YX-02-030 and detection of MDM2. A. Chemical synthesis of the MDM2-PROTAC YX-02-030. B. Surface plasmon resonance (SPR) kinetic plots for the compounds indicated. Graphical data is a representative of a single experiment which was repeated three times with similar results. C.

Western blots for MDM2 using multiple antibodies in MDA-MB-231 cells treated with MDM2-PROTAC (6 μM, 16 hr). D. qRT-PCR (quadruplicate) for MDM2 expression in MDA-MB-231 and MDA-MB-436 cells treated with MDM2-PROTAC (6 μM) or DMSO vehicle control for the indicated times; mean±SEM. MCF7 breast cancer cells treated with etoposide (10 μM, 24 hr) served as a positive control for MDM2 expression. Data from each cell line was made relative to its own DMSO control; *P<0.0001 (1-way ANOVA), comparing MDM2-PROTAC or etoposide to DMSO vehicle control.

FIGS. 21A-21E show the apoptosis induction of p53-inactivated TNBC cells in 2D and 3D cultures by the MDM2-PROTAC YX-02-030. A-B. Colony formation assays (A. Triplicate, 12 days) and mammosphere formation (B. Triplicate 72 hrs) of MDA-MB-231 and MDA-MB-436 cells in the presence of the indicated compounds or DMSO control (−); representative pictures shown. C-E. Formed mammospheres of MDA-MB-231 and MDA-MB-436 cells were treated with the compounds indicated or DMSO control (−), and area (C. n=10 mammospheres, relative to pre-treatment area, 72 hr), real-time imaging (IncuCyte) and quantification of Caspase-3/7 activity (D. n=4-5 wells/compound), and survival/ATP assays (E. Quadruplicate, 72 hr) were performed; representative images shown; scale bar 300 m. Mean±SEM (A-C and E). For A, *P<0.0001 and **P<0.0001, comparing 1 M and 2 M PROTAC, respectively, to control compounds and DMSO vehicle control. For B, *P<0.0046, **P<0.0001, and ***P<0.0001, comparing 0.5 M, 1 M, and 2 M PROTAC, respectively, to control compounds and DMSO vehicle control. For C, *P<0.0001 and **P<0.0001, comparing 2 M and 4 M/6 M PROTAC, respectively, to control compounds and DMSO vehicle control. For E, *P<0.0001 and **P<0.0001, comparing 3 M and 4 M PROTAC, respectively, to control compounds and DMSO vehicle control. 1-way ANOVA (A-C and E).

FIGS. 22A & 22B show that the Mdm2 PROTACs YX-02-030 and YX-02-228 are cell permeable, are active in cell-based assays (as shown by degradation and effects on cell viability) and possess suitable physiochemical properties predictive of reasonable pharmacokinetic exposure in vivo for translation to in vivo efficacy studies. A. Half-life of parent remaining and intrinsic clearance (CLint) determined from the first-order elimination constant by nonlinear regression. B. YX-02-030 and YX-02-228 with prolonged in vivo plasma exposure.

FIGS. 23A-23G show that MDM2-PROTAC YX-02-030 kills p53-inactivated TNBC cells in vivo. A-G. MDA-MB-231 and MDA-MB-436 cells were injected (subcutaneous) into one flank of nude mice and allowed to form palpable tumors (approximately 80 mm³). Mice (randomized into tumor-size matched groups) were treated (intraperitoneal injection) with MDM2-PROTAC YX-02-030, RG7112D, or vehicle control at 50 mg/kg once daily for 14 days (treatment start, green arrow and stop, red arrow). Kaplan-Meier survival analysis (A) and tumor volume (B; individual, left and averaged, right) were evaluated overtime. C-G, After 72 hr of treatment, tumors (n=3/group; −, vehicle control) were harvested and protein expression evaluated by Western blotting (C), Annexin-V positivity (D), Caspase-3 activity (E), apoptotic subG1 DNA content (F), and viability by Trypan Blue dye exclusion (G). Mean±SD (B (right), D-G). Log-rank tests for A, and longitudinal tumor growth analysis using 2-way ANOVA with Bonferroni correction for B; P-values indicated. *P<0.0021 for D, *P<0.0001 for E, *P<0.0147 for F, and *P<0.0014 for G were determined using unpaired two-tailed t-tests, comparing PROTAC to vehicle control.

FIGS. 24A-24E show no overt toxicities from the MDM2-PROTAC YX-02-030 observed in vivo. A-B. A. After C57Bl/6 immunocompetent mice received 15 consecutive intraperitoneal (IP) injections of MDM2-PROTAC (50 mg/kg) or vehicle control treatments, complete blood counts were determined. B. H&E sections of formalin-fixed paraffin-embedded tissues evaluated (B, BM-bone marrow). C-E, Nude mice with subcutaneous tumors of MDA-MB-231 and MDA-MB-436 TNBC cells were administered (IP) MDM2-PROTAC YX-02-030, RG7112D, or vehicle control (50 mg/kg) for 14 consecutive days. C. Mouse weight was measured daily throughout the course of treatment. D. Complete blood counts were determined following 13 consecutive treatments. E. Sections of formalin-fixed paraffin-embedded tissues from mice at time of sacrifice were H&E stained and evaluated. Representative images are shown and scale bars are 200 m (spleen) and 300 μm (bone marrow and intestine). Mean±SD (A, C, D).

FIGS. 25A & 25B show the in vivo tolerability tests conducted with the 2 lead Mdm2 PROTACs to evaluate tolerated doses and effects on normal tissues. Nude mice were treated with Mdm2 PROTACs, YX-02-030 (50 mg/kg; IP) and YX-02-228 (30 mg/kg; IP) once daily for 14 days. A-B. Results showed no adverse clinical observations for spleen and bone marrow FIG. 26 shows the effects of YX-02-030 on TNBC patient samples. Fresh, surgically-resected tumor from five TNBC patients was obtained, and 80% were from Black/African American patients.

FIGS. 27A-27E show TP53 mutant TNBC patient-derived explants undergo apoptosis following treatment with the MDM2-PROTAC YX-02-030. A. TP53 sequenced in five fresh TNBC patient samples and used in explant and 3D mammosphere cultures. B-C. TNBC patient-derived explants were treated with the compounds indicated and Western blotting (B) and immunohistochemistry (IHC) of FFPE sections for cleaved Caspase-3 (C, n=3 paired patient samples) were performed. Representative images of H&E and cleaved Caspase-3 (CC3) IHC shown, including normal breast epithelium treated with MDM2-PROTAC YX-02-030; scale bar 300 m. D-E. Mammosphere cultures were established from TNBC patient samples, treated with the indicated compounds, and live-cell detection of Caspase-3/7 activity (D, quadruplicate) and mammosphere survival/ATP production (E, quadruplicate) were measured.

Normal breast epithelium from a patient that formed loose aggregates was also treated with MDM2-PROTAC YX-02-030. For comparison, values for TNBC-4 and the normal breast mammospheres are relative to DMSO vehicle control (E, right graph). Representative images shown (RG, RG7112 and RG-D, RG7112D); scale bar 300 m. Mean±SD (C-E). For C, *P=0.0477 (unpaired two-tailed i-test), comparing YX-02-030 to DMSO. For D and E, *P<0.0001 (2-way and 1-way ANOVA, respectively), comparing YX-02-030 to control compounds and DMSO vehicle control.

FIGS. 28A-28L show TAp73 is activated by the MDM2-PROTAC YX-02-030 and mediates apoptosis of p53-inactivated TNBC cells. A-C, RNA-seq (triplicates) was performed on MDA-MB-231 cells treated with the MDM2-PROTAC YX-02-030 (6 μM, 16 hr) or DMSO vehicle control or expressing MDM2 shRNA or non-targeting control shRNA (48 hr). A, Pathway analysis using Hallmark gene signatures. B, Heatmap of p53 family target genes of individual samples (3 each). C, Spearman's correlation of differentially expressed p53 family target genes between PROTAC-treated relative to DMSO vehicle control and MDM2 shRNA relative to non-targeting shRNA control; Spearman's correlation coefficient (p) and P-value indicated. D, qRT-PCR analysis (triplicate, 6 μM, 16 hr) to validate RNA-seq results. E-G, Western blotting performed for the proteins indicated following treatment with MDM2-PROTAC YX-02-030, control compounds, or DMSO vehicle control (−) of MDA-MB-231 cells growing in culture (E, 6 μM, 16 hr), MDA-MB-231 subcutaneous tumors harvested 72 hr after treatment began (G, left), TNBC patient-derived explants (G, right), and following knockdown of MDM2 with two shRNA or non-targeting shRNA (shNT) control in MDA-MB-231 cells (F). H, MDA-MB-231 cells pre-treated (1 hr) with MG132 were treated with MDM2-PROTAC YX-02-030 (6 μM, 16 hr) or DMSO vehicle control and MDM2 (left) and TAp73 (right) were immunoprecipitated and proteins Western blotted. I, ChIP for TAp73 was performed with MDA-MB-231 cells following treatment with the MDM2-PROTAC YX-02-030 (6 μM, 16 hr) or DMSO vehicle control, and enrichment of TAp73 (triplicate; first normalized to input DNA then IgG control) was determined at the loci indicated. J and K, After 48 hr, MDA-MB-231 cells expressing two independent TAp73 shRNA or non-targeting shRNA (shNT) control were treated with MDM2-PROTAC YX-02-030 or DMSO vehicle control (6 μM, 16 hr). p53/TAp73 target genes were evaluated by Western blotting (J) and qRT-PCR (K, triplicate). L, MTT assay (quadruplicate, 24 hr intervals) of MDA-MB-231 cells expressing two independent TAp73 shRNA or non-targeting shRNA (shNT) control and treated with MDM2-PROTAC YX-02-030 (4 μM) or DMSO vehicle control. Mean±SEM (D, I, K) and mean±SD (L). For D and I, *P<0.0001 (unpaired two-tailed t-tests), comparing PROTAC to DMSO vehicle control or MDM2 shRNA to non-targeting control shRNA. For K, *P<0.0015, comparing non-targeting sbRNA control with PROTAC YX-02-030 to DMSO vehicle control and **P<0.0012, comparing TAp73 shRNA with PROTAC YX-02-030 to non-targeting shRNA control with PROTAC YX-02-030; 2-way ANOVA. For L, *P<0.00081, comparing non-targeting control shRNA with PROTAC YX-02-030 to DMSO vehicle control and **P<0.0031, comparing TAp73 shRNA with PROTAC YX-02-030 to non-targeting control shRNA with PROTAC YX-02-030; 2-way ANOVA. For E-G and J, long exp is long exposure.

FIGS. 29A-29D show that p53 family target genes are up-regulated following MDM2 degradation or MDM2 knockdown. RNA-seq (triplicates) was performed on MDA-MB-436 cells treated with MDM2-PROTAC YX-02-030 (6 μM, 16 hr) or DMSO vehicle control and cells expressing MDM2 shRNA or non-targeting control shRNA (48 hr). A, Pathway analysis using Hallmark gene signatures. B. Heatmap of p53 family target genes of individual samples (3 samples per group). C. Spearman's correlation of differential p53 family target gene expression between PROTAC-treated relative to DMSO vehicle control and MDM2 shRNA relative to non-targeting shRNA control; Spearman's correlation coefficient (r) and P-value are indicated. D. qRT-PCR analysis (triplicate, 6 μM, 16 hr) to validate RNA-seq results; mean±SEM. For D, *P<0.0001 using unpaired two-tailed t-tests, comparing MDM2-PROTAC YX-02-030 to DMSO vehicle control or MDM2 shRNA to non-targeting control shRNA.

FIGS. 30A-30J show that MDM2-PROTAC YX-02-030 activates TAp73, which is then required to mediate apoptosis of p53-inactivated TNBC cells. A-C. Western blotting was performed for the proteins indicated following treatment with MDM2-PROTAC YX-02-030, control compounds, or DMSO vehicle control (−) of MDA-MB-436 cells growing in culture (A, 6 μM, 16 hr), MDA-MB-436 subcutaneous tumors harvested 72 hr after treatment began (C, right; MDM2 blots are same as in FIG. 25E), and in 3 additional TNBC cell lines (C, left), and following knockdown of MDM2 with two shRNA or non-targeting shRNA (shNT) control (B). D. Western blots comparing lysis methods used to extract proteins. E, qRT-PCR (triplicate) for TAp73 following treatment of MDA-MB-231 and MDA-MB-436 cells with MDM2-PROTAC YX-02-030 (6 μM, 16 hr) or DMSO vehicle control or cells expressing MDM2 shRNA or non-targeting control shRNA (48 hr). Cells treated with etoposide (10 μM, 24 hr) were a positive control for TAp73 expression. F. MDA-MB-436 cells pre-treated with MG132 for 1 hr were treated with MDM2-PROTAC YX-02-030 (6 μM, 16 hr) or DMSO vehicle control and MDM2 (top) and TAp73 (bottom) were immunoprecipitated and Western blots performed. G, ChIP for TAp73 was performed with MDA-MB-436 cells following treatment with the PROTAC (6 μM, 16 hr) or DMSO vehicle control and enrichment of TAp73 (triplicate; first normalized to input DNA then IgG control) was determined at the loci indicated. H-I. MDA-MB-436 cells expressing two independent TAp73 shRNA or nontargeting shRNA (shNT) control were treated with MDM2-PROTAC YX-02-030 or DMSO vehicle control (6 μM, 16 hr). p53/TAp73 target genes were evaluated by Western blotting (H) and qRT-PCR (I, triplicate). J. MTT assay (quadruplicate, every 24 hr) of MDA-MB-436 cells with two independent TAp73 shRNA or non-targeting shRNA (shNT) control treated with MDM2-PROTAC YX-02-030 or DMSO vehicle control (4 μM). Mean±SEM (E, G, I) and mean±SD (J). For E, *P<0.0001 (1-way ANOVA), comparing MDM2-PROTAC YX-02-030 or etoposide to DMSO vehicle control or MDM2 shRNA to non-targeting control shRNA. For G, *P<0.0001 (unpaired two-tailed t-tests), comparing YX-02-030 to DMSO vehicle control. For I, *P<0.0034, comparing non-targeting control shRNA with YX-02-030 to DMSO vehicle control and **P<0.0039, comparing TAp73 shRNA with YX-02-030 to non-targeting shRNA control with YX-02-030; 2-way ANOVA. For J, *P<0.0001, comparing non-targeting control shRNA with YX-02-030 to DMSO vehicle control and **P<0.0099, comparing TAp73 shRNA with YX-02-030 to non-targeting shRNA control with YX-02-030; 2-way ANOVA. For A, B, and H, long exp is long exposure.

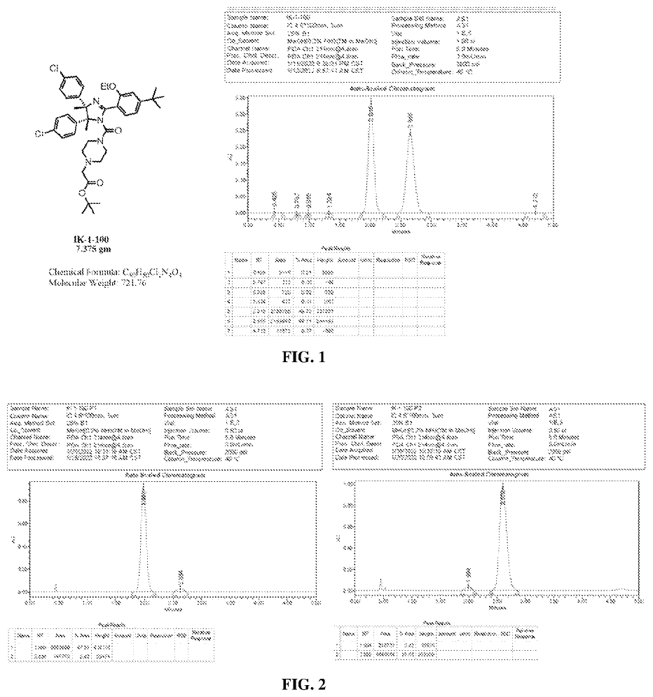

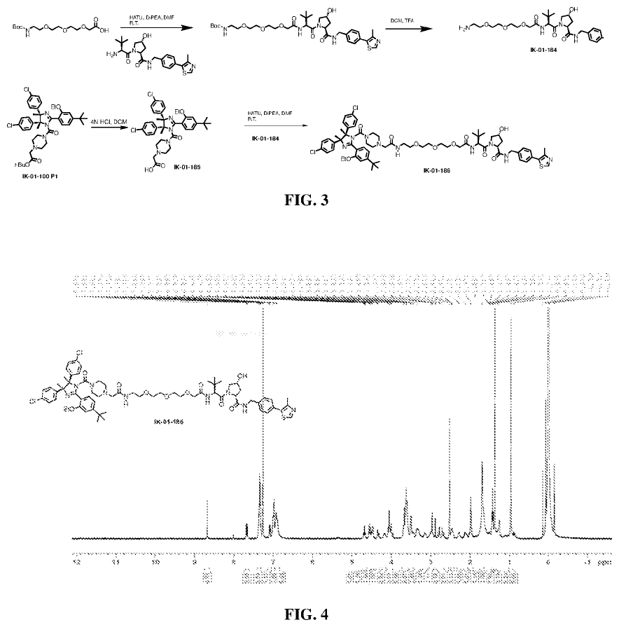

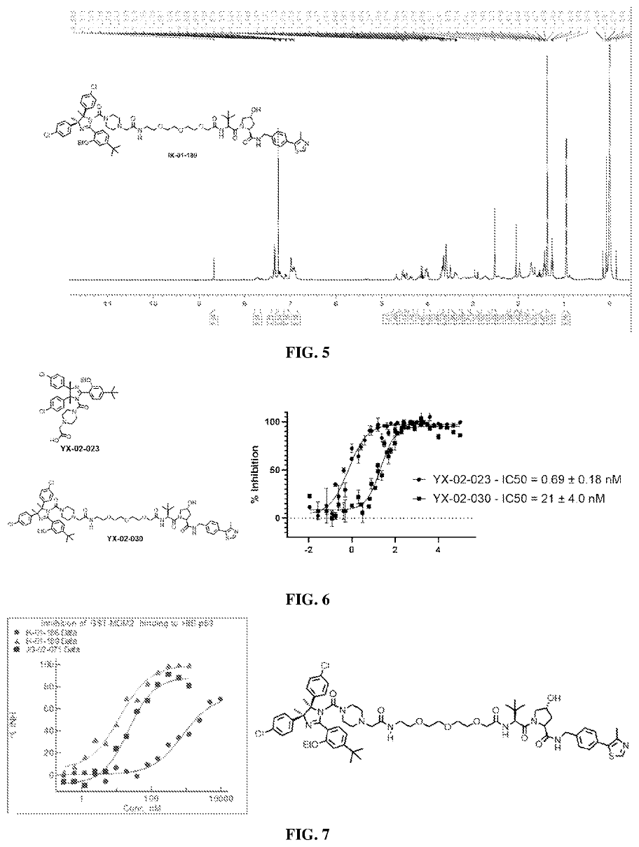

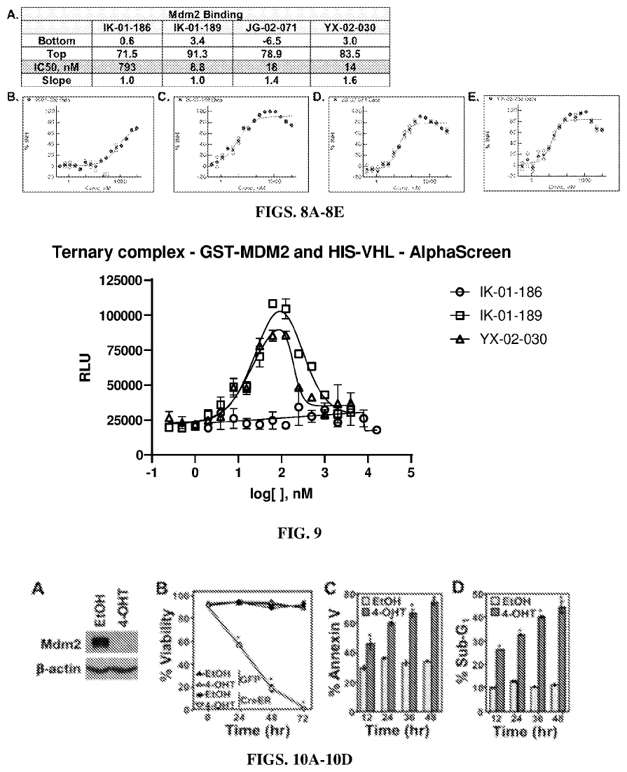

### DESCRIPTION OF ILLUSTRATIVE EMBODIMENTS

Provided herein are compounds that may be used to degrade Mdm2 and thus may be used in the treatment of cancers including cancers associated with overexpression of Mdm2 and/or cancers which may be treated by modulating the activity of p53. These compounds may show improved tolerability and stability compared to traditional inhibitors of Mdm2. These and more details will be described below.

### I. Compounds and Formulations Thereof

A. Compounds of the Present Disclosure The compounds of the present disclosure are shown, for example, in the summary section above and in Table 1 and the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Compounds of the Present Disclosure

| Com- | |
|---|---|
| pound | |
| ID | Structure |
| YX-02- 020 | 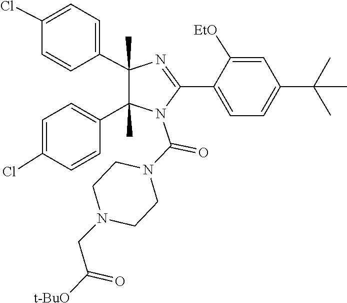 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)OC(C)(C)C)CC1] |
| YX-02- 023 | 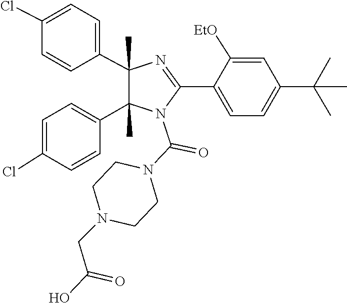 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1] |
| YX-02- 030 | 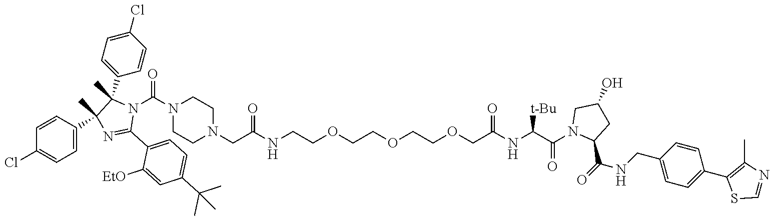 [SMILES: CCOc1cc(C(C)(C)C)ccc1C1=N[C@](C)(c2ccc(Cl)cc2)[C@](C)(c2ccc(Cl)cc2)N1C(=O)N1CCN(CC(=O)NCCOCCOCCOCC(=O)N[C@H](C(=O)N2C[C@H](O)C[C@H]2C(=O)NCc2ccc(-c3scnc3C)cc2)C(C)(C)C)CC1] |
| YX-02- 041 | 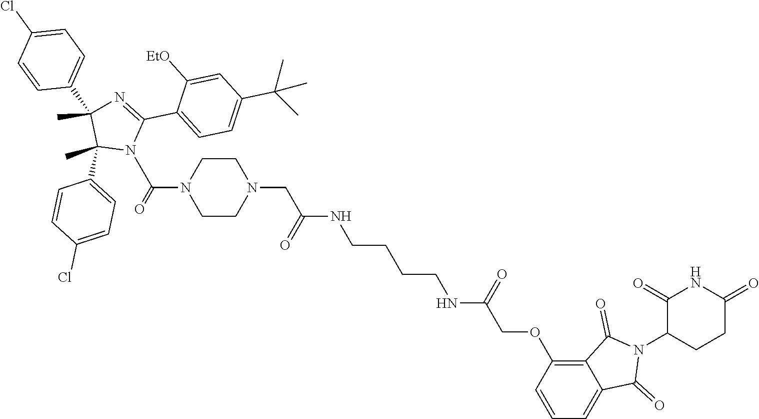 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCCCNC(=O)COc2cccc3c2C(=O)N(C2CCC(=O)NC2=O)C3=O)CC1] |
| YX-02- 052 | 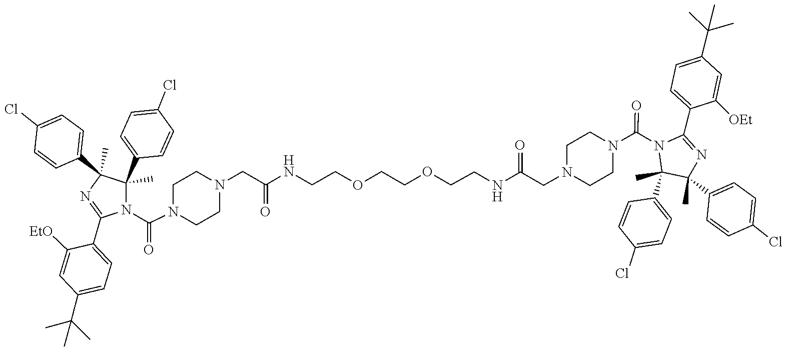 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCOCCOCCNC(=O)CN2CCN(C(=O)N3C(c4ccc(C(C)(C)C)cc4OCC)=N[C@](C)(c4ccc(Cl)cc4)[C@]3(C)c3ccc(Cl)cc3)CC2)CC1] |
| YX-02- 062 | 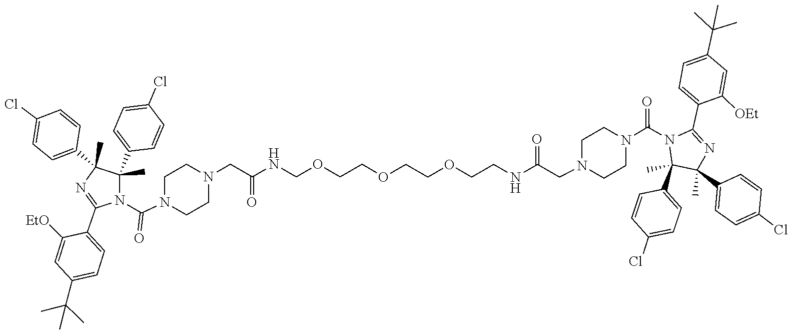 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCOCCOCCOCNC(=O)CN2CCN(C(=O)N3C(c4ccc(C(C)(C)C)cc4OCC)=N[C@](C)(c4ccc(Cl)cc4)[C@]3(C)c3ccc(Cl)cc3)CC2)CC1] |
| YX-02- 230 | 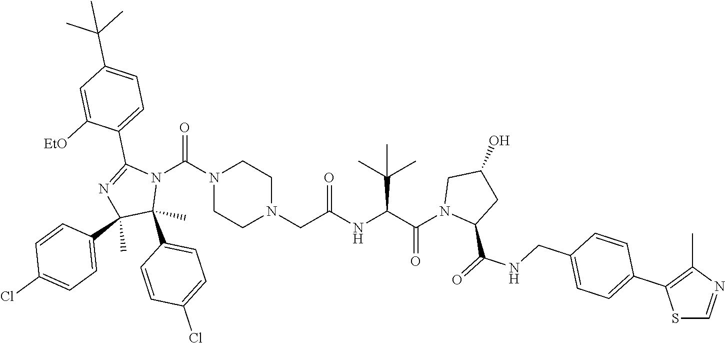 [SMILES: CCOc1cc(C(C)(C)C)ccc1C1=N[C@](C)(c2ccc(Cl)cc2)[C@](C)(c2ccc(Cl)cc2)N1C(=O)N1CCN(CC(=O)N[C@H](C(=O)N2C[C@H](O)C[C@H]2C(=O)NCc2ccc(-c3scnc3C)cc2)C(C)(C)C)CC1] |
| YX-02- 219 | 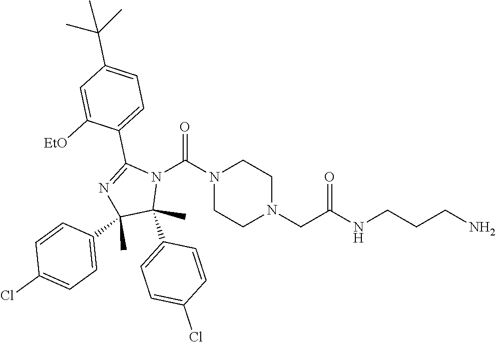 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCCN)CC1] |
| YX-02- 228 | 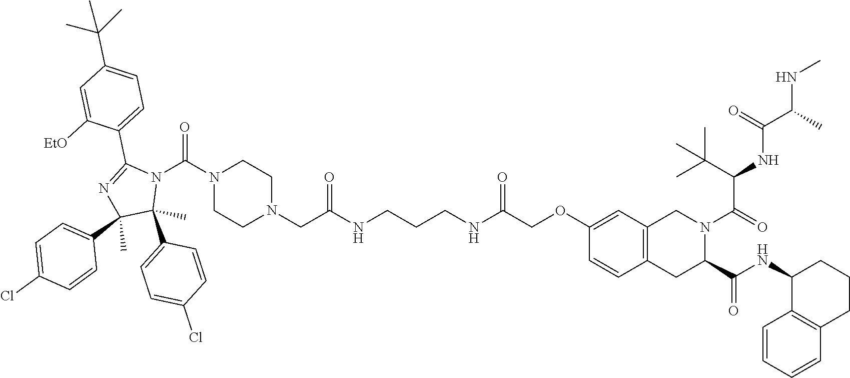 [SMILES: CCOc1cc(C(C)(C)C)ccc1C1=N[C@](C)(c2ccc(Cl)cc2)[C@](C)(c2ccc(Cl)cc2)N1C(=O)N1CCN(CC(=O)NCCCNC(=O)COc2ccc3c(c2)CN(C(=O)[C@H](NC(=O)[C@@H](C)NC)C(C)(C)C)[C@@H](C(=O)N[C@H]2CCCc4ccccc42)C3)CC1] |

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### B. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$HED ( mg / kg ) = Animal dosee ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount is from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### II. Methods of Treatment and Combination Therapies

### A. Methods of Treatment

In particular the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, the amount of the compounds used is calculated to be from about 0.01 mg to about 10,000 mg/day. In some embodiments, the amount is from about 1 mg to about 1,000 mg/day. In some embodiments, the compounds may be administered for 1 day to 20 days. In further embodiments, it is contemplated that the compounds may be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days, or any range derivable therein. In some embodiments, the compounds may be administered for between 3 and 5 days, inclusive. In some embodiments, the compounds may be administered once. It is also contemplated that in some embodiments, the compounds disclosed herein may be administered two or more times. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### B. Hyperproliferative Diseases

While hyperproliferative diseases can be associated with any medical disorder that causes a cell to begin to reproduce uncontrollably, the prototypical example is cancer. One of the key elements of cancer is that the normal apoptotic cycle of the cell is interrupted and thus agents that lead to apoptosis of the cell are important therapeutic agents for treating these diseases. As such, the Md2 degraders described in this disclosure may be effective in treating cancers. In particular, the Mdm2 degraders may be used to treat one or more cancers that expresses a mutant p53 gene or that have deleted or truncated the p53 gene. The p53 gene may be mutated in such a way that it results in decreased expression, decreased activity, or is non-functional. Similarly, the p53 gene may also be the wild type gene. In another embodiments, the p53 gene in the cancer may be null. In particular, the present disclosure relates to the treatment of one or more cancers such as breast cancer including triple negative breast cancer (TNBC). The present disclosure also relates to the treatment of lymphoma such as Burkitt lymphoma, Diffuse large B cell lymphoma, and T cell lymphoma. The present disclosure also relates to the treatment of leukemia such as B cell acute lymphoblastic leukemia (B-ALL) or T cell acute lymphoblastic leukemia (T-ALL). The present disclosure also relates to the treatment of lung cancer such as lung adenocarcinoma. The present disclosure also relates to the treatment of ovarian cancer, head and neck cancer, or sarcoma.

Cancer cells that may be treated with the compounds according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### C. Combination Therapies

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

To treat diseases or disorders using the methods and compositions of the present disclosure, one would generally contact a cell or a subject with a compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent. In some embodiments, the compounds of the present disclosure or any therapies used in conjunction with the compounds of the present disclosure may be administered in a less than therapeutically effective dose when used either alone or in combination.

Non-limiting examples of such combination therapy include combination of one or more compounds of the invention with another pro-inflammatory agent, an immunosuppressant agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

It is also conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are also contemplated.

### III. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “−” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

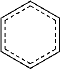

covers, for example

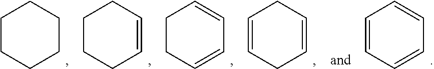

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

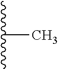

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry [symbol] around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

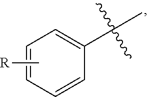

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

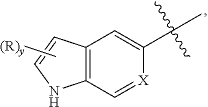

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C=8)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n +2 electrons in a fully conjugated cyclic 11 system.

An aromatic compound or chemical group may be depicted as a single resonance structure; however, depiction of one resonance structure is taken to also refer to any other resonance structure. For example:

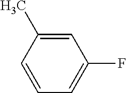

is also taken to refer to

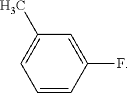

Aromatic compounds may also be depicted using a circle to represent the delocalized nature of the electrons in the fully conjugated cyclic a system, two non-limiting examples of which are shown below;

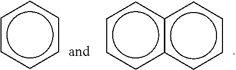

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

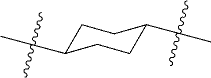 [SMILES: *C1CCC(*)CC1]

is a non-limiting example of cycloalkanediyl group. A “cycloalkane” refers to the class of compounds having the formula H-R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” refers to a divalent unsaturated aliphatic group, with two carbon atoms as points of attachment, a linear or branched acyclic structure, at least one nonaromatic carbon-carbon triple bond, no carbon-carbon double bonds, and no atoms other than carbon and hydrogen. The groups —C≡C—, —C≡CCH₂—, and —CH₂C═CCH₂— are non-limiting examples of alkynediyl groups. It is noted that while the alkynediyl group is aliphatic, once connected at both ends, this group is not precluded from forming part of an aromatic structure. An “alkyne” refers to the class of compounds having the formula H-R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

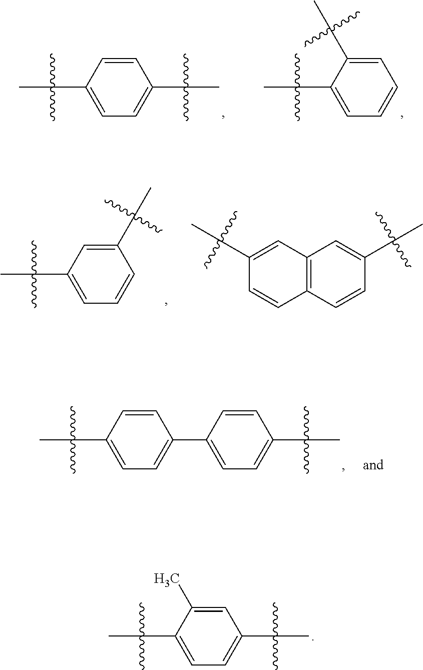

An “arene” refers to the class of compounds having the formula H-R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃.

An “amine protecting group” or “amino protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxy-benzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monvalently protected amino group” and PG_{DA} is a divalent amine protecting group as described above, which may also be described as a “divalently protected amino group”.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —C02H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

As used herein, the term “functional group” refers to any chemical group or substituent covalently bound to a core structure. Functional groups may include but are not limited to hydrogen, alkyl, alkenyl, alkynyl, cycloalkyl, heterocycloalkyl, aryl, heteroaryl, aralkyl, —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, —S(O)₂NH₂ or a combination or substituted version of any of these groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility.

Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains <15%, more preferably <10%, even more preferably <5%, or most preferably <1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “ubiquitin ligase ligand” or “E3 ligase ligand” refers to a chemical group capable of binding ubiquitin ligase. Ubiquitin ligase (also called an E3 ubiquitin ligase) is a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. E3 ligases interact with both the target protein and the E2 enzyme, and so impart substrate specificity to the E2. Commonly, E3s polyubiquitinate their substrate with Lys48-linked chains of ubiquitin, targeting the substrate for destruction by the proteasome.

However, one of skill in the art recognizes that many other types of linkages are possible and that each may alter a protein's activity, interactions, or localization. Ubiquitination by E3 ligases regulates diverse areas such as cell trafficking, DNA repair, and signaling and is of profound importance in cell biology. E3 ligases are also key players in cell cycle control, mediating the degradation of cyclins, as well as cyclin dependent kinase inhibitor proteins. The human genome encodes over 600 putative E3 ligases, allowing for tremendous diversity in substrates. Non-limiting examples of ubiquitin ligase ligands include the von Hippel-Lindau (VHL) ligand, a cIAP1 ligand, a MDM2 ligand, a CRBN ligand, a CUL2 ligand, or other ligand which binds to one or more of the proteins of the ubiquitin protein complex especially the E3 component of this complex.

Ubiquitin ligase (also called an E3 ubiquitin ligase or E3 ligase) is a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. E3 ligases interact with both the target protein and the E2 enzyme, and so impart substrate specificity to the E2. Commonly, E3s polyubiquitinate their substrate with Lys48-linked chains of ubiquitin, targeting the substrate for destruction by the proteasome. However, one of skill in the art recognizes that many other types of linkages are possible and that each may alter a protein's activity, interactions, or localization. Ubiquitination by E3 ligases regulates diverse areas such as cell trafficking, DNA repair, and signaling and is of profound importance in cell biology. E3 ligases are also key players in cell cycle control, mediating the degradation of cyclins, as well as cyclin dependent kinase inhibitor proteins. The human genome encodes over 600 putative E3 ligases, allowing for tremendous diversity in substrates. Non-limiting examples of ubiquitin ligase ligands include the von Hippel-Lindau (VHL) ligand, a cIAP1 ligand, a MDM2 ligand, a CRBN ligand, a CUL2 ligand, or other ligand which binds to one or more of the proteins of the ubiquitin protein complex especially the E3 component of this complex.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### I. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synthetic Methodology and Characterization Data

### A. Synthesis of MDM2 PROTACs

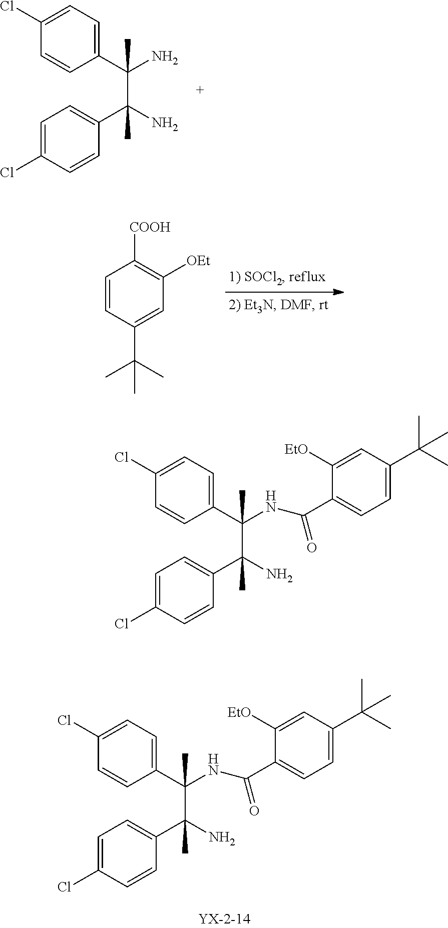 [SMILES: C[C@](N)(c1ccc(Cl)cc1)[C@](C)(N)c1ccc(Cl)cc1 ; CCOc1cc(C(C)(C)C)ccc1C(=O)O ; CCOc1cc(C(C)(C)C)ccc1C(=O)N[C@@](C)(c1ccc(Cl)cc1)[C@](C)(N)c1ccc(Cl)cc1 ; CCOc1cc(C(C)(C)C)ccc1C(=O)N[C@@](C)(c1ccc(Cl)cc1)[C@](C)(N)c1ccc(Cl)cc1]

N-((2S,3R)-3-amino-2,3-bis(4-chlorophenyl)butan-2-yl)-4-(tert-butyl)-2-ethoxybenzamide (YX-02-014): 4-(tert-butyl)-2-ethoxy-benzoyl acid (666 mg, 3.0 mmol) was dissolved in toluene and treated with oxalyl chloride (320 μL). After it was stirred at 50° C. for 1 h, the reaction solvent was removed and used directly for next step. A mixture of the obtained product and triethylamine (823 μL, 6.0 mmol) in DCM (15 mL) was reacted with 2,3-bis-(4-chlorophenyl)-2,3-butanediamine (840 mg, 3.0 mmol) for 30 min at room temperature. The mixture was partitioned between 10% sodium bicarbonate solution and dichloromethane. The organic phase was separated and washed with water, dried over anhydrous sodium sulfate, concentrated and purified by chromatography on silica gel to afford YX-02-014 (600 mg, 43% yield).

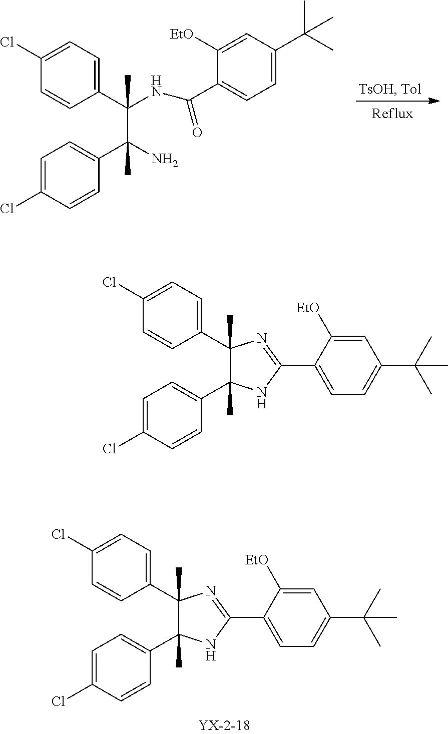 [SMILES: CCOc1cc(C(C)(C)C)ccc1C(=O)N[C@@](C)(c1ccc(Cl)cc1)[C@](C)(N)c1ccc(Cl)cc1 ; CCOc1cc(C(C)(C)C)ccc1C1=N[C@@](C)(c2ccc(Cl)cc2)[C@@](C)(c2ccc(Cl)cc2)N1 ; CCOc1cc(C(C)(C)C)ccc1C1=N[C@@](C)(c2ccc(Cl)cc2)[C@@](C)(c2ccc(Cl)cc2)N1]

(4S,5R)-2-(4-(tert-butyl)-2-ethoxyphenyl)-4,5-bis(4-chlorophenyl)-4,5-dimethyl-4,5-dihydro-1H-imidazole (YX-02-018): N-((2S,3R)-3-amino-2,3-bis(4-chlorophenyl)butan-2-yl)-4-(tert-butyl)-2-ethoxybenzamide (YX-02-014) (300 mg, 0.58 mmol) was dissolved in Tol (15 mL) and treated with TsOH (20 mg, 0.116 mmol) and heat to reflux for 4 h, then remove the solvent, purified by chromatography on silica gel to afford YX-02-018 (220 mg, 73% yield).

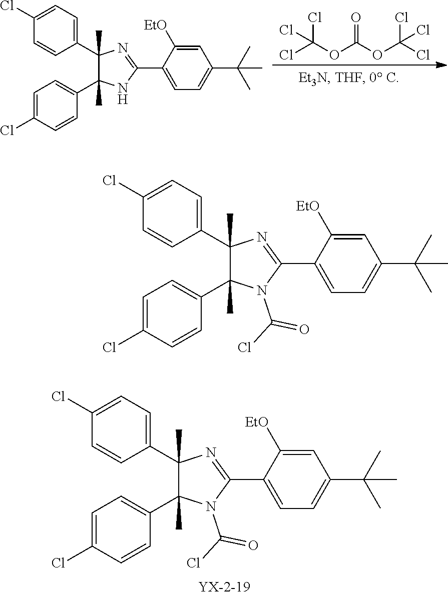

(4S,5R)-2-(4-(tert-butyl)-2-ethoxyphenyl)-4,5-bis(4-chlorophenyl)-4,5-dimethyl-4,5-dihydro-1H-imidazole-1-carbonyl chloride (YX-02-019): To a solution of (4S,5R)-2-(4-(tert-butyl)-2-ethoxyphenyl)-4,5-bis(4-chlorophenyl)-4,5-dimethyl-4,5-dihydro-1H-imidazole (YX-02-018) (220 mg, 0.44 mmol) and triethylamine (300p L, 2.2 mmol) in DCM (15 mL) at 0° C. was added triphosgene (600 mg, 2.0 mmol). After stirred for 30 min the mixture was taken up and washed with water, dried over anhydrous sodium sulfate, concentrated and purified by chromatography on silica gel to afford YX-02-019 (200 mg, 81% yield).

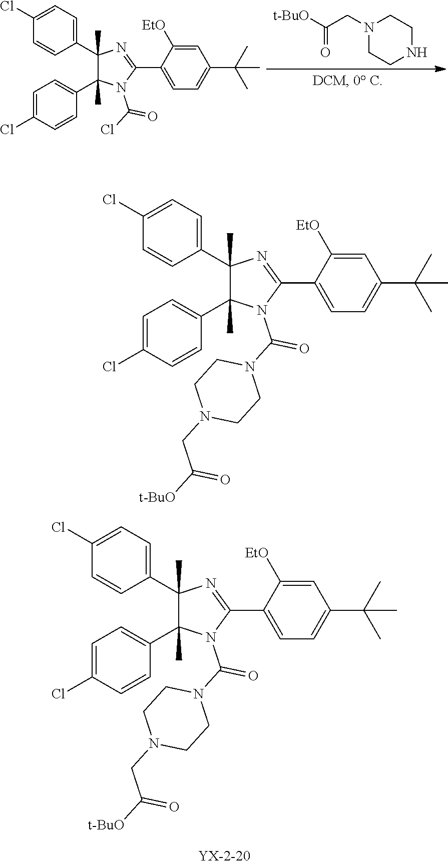 [SMILES: CCOc1cc(C(C)(C)C)ccc1C1=N[C@@](C)(c2ccc(Cl)cc2)[C@@](C)(c2ccc(Cl)cc2)N1C(=O)Cl ; CC(C)(C)OC(=O)CN1CCNCC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)OC(C)(C)C)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)OC(C)(C)C)CC1]

Tert-butyl-2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetate (YX-02-020): To a solution of (4S,5R)-2-(4-(tert-butyl)-2-ethoxyphenyl)-4,5-bis(4-chlorophenyl)-4,5-dimethyl-4,5-dihydro-1H-imidazole-1-carbonyl chloride (YX-02-019) (120 mg, 0.214 mmol) and tert-butyl 2-(piperazin-1-yl) acetate (80 mg, 0.428 mmol) in DCM (10 mL) at 0° C. was added triethylamine (240 μL, 2 mmol). The reaction was allowed to react for 24 hour. The mixture was partitioned between 10% sodium bicarbonate solution and dichloromethane. The organic phase was separated and washed with water, dried over anhydrous sodium sulfate, concentrated, and purified by chromatography on silica gel to afford YX-02-020 (150 mg, 99% yield).

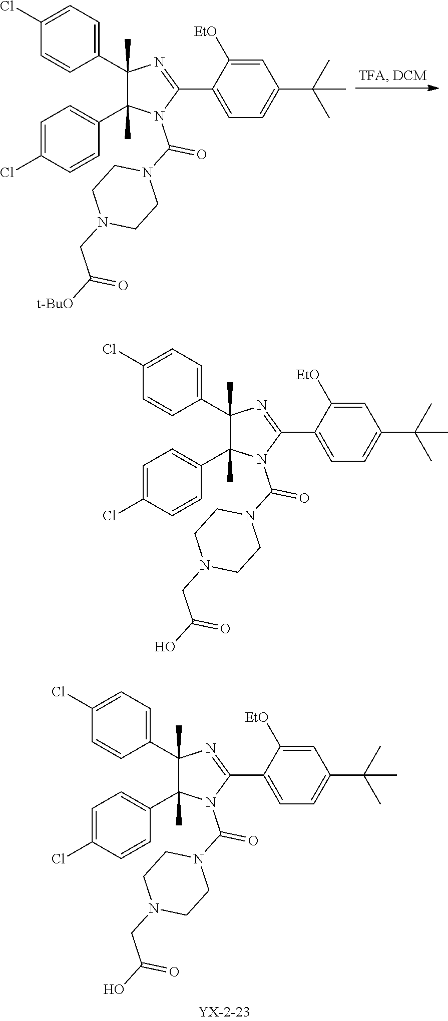 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)OC(C)(C)C)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1]

2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (YX-02-023): Tert-butyl-2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetate (YX-02-020) (150 mg, 0.2 mmol) was dissolved in DCM (5 mL) and treated with TFA (1 mL), after stirred for 18 hour, remove the reaction solvent and use directly for next step.

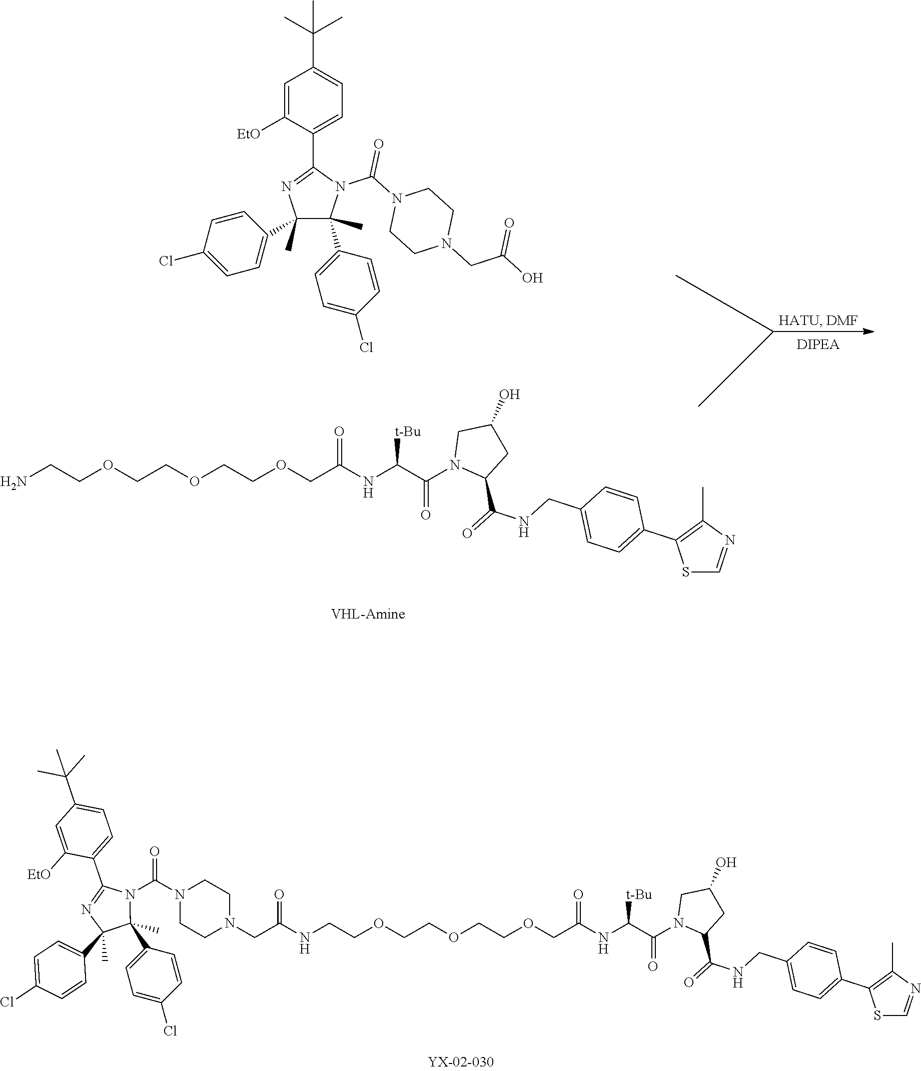

(2S,4R)-1-((S)-2-(tert-butyl)-17-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)-4,16-dioxo-6,9,12-trioxa-3,15-diazaheptadecanoyl)-4-hydroxy-N-(4-(4-methylthiazol-5-yl)benzyl)pyrrolidine-2-carboxamide (YX-02-030): A mixture of VHL-Amine (31 mg, 0.05 mmol) and 2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (YX-02-020) (36 mg, 0.05 mmol) in DMF (3 mL) was treated with DIPEA (16 μL, 0.1 mmol) and then HATU (20 mg, 0.05 mmol) and stirred at ambient temperature for 1 hour. After which time the reaction mixture was diluted with H₂O and extracted with EtOAc. The organic layer was dried by Na₂SO₄ and concentrated in vacuum. The resulting material was purified by chromatography on silica to afford YX-02-030 (28 mg, 50% yield). ¹H NMR (400 MHz, DMSO) δ 8.98 (s, 1H), 8.59 (s, 1H), 7.58 (d, J=8.2 Hz, 2H), 7.48-7.32 (m, 5H), 7.05 (dd, J=24.6, 15.3 Hz, 9H), 5.15 (s, 1H), 4.56 (d, J=9.5 Hz, 1H), 4.48-4.38 (m, 2H), 4.35 (s, 2H), 4.25 (d, J=11.5 Hz, 2H), 3.96 (s, 3H), 3.67 (d, J=7.6 Hz, 1H), 3.59 (s, 3H), 3.56 (s, 2H), 3.51 (d, J=3.7 Hz, 2H), 3.49 (s, 2H), 3.36 (d, J=6.1 Hz, 2H), 3.17 (d, J=6.2 Hz, 3H), 2.60 (s, 2H), 2.44 (s, 4H), 2.21 (s, 1H), 2.04 (d, J=8.6 Hz, 1H), 1.88 (s, 4H), 1.59 (s, 4H), 1.38 (d, J=16.7 Hz, 9H), 1.28 (dd, J=15.2, 8.1 Hz, 8H), 0.94 (s, 9H), 0.90-0.77 (m, 5H). LC-MS RT=1.81, [(M+2H)/2]=634.

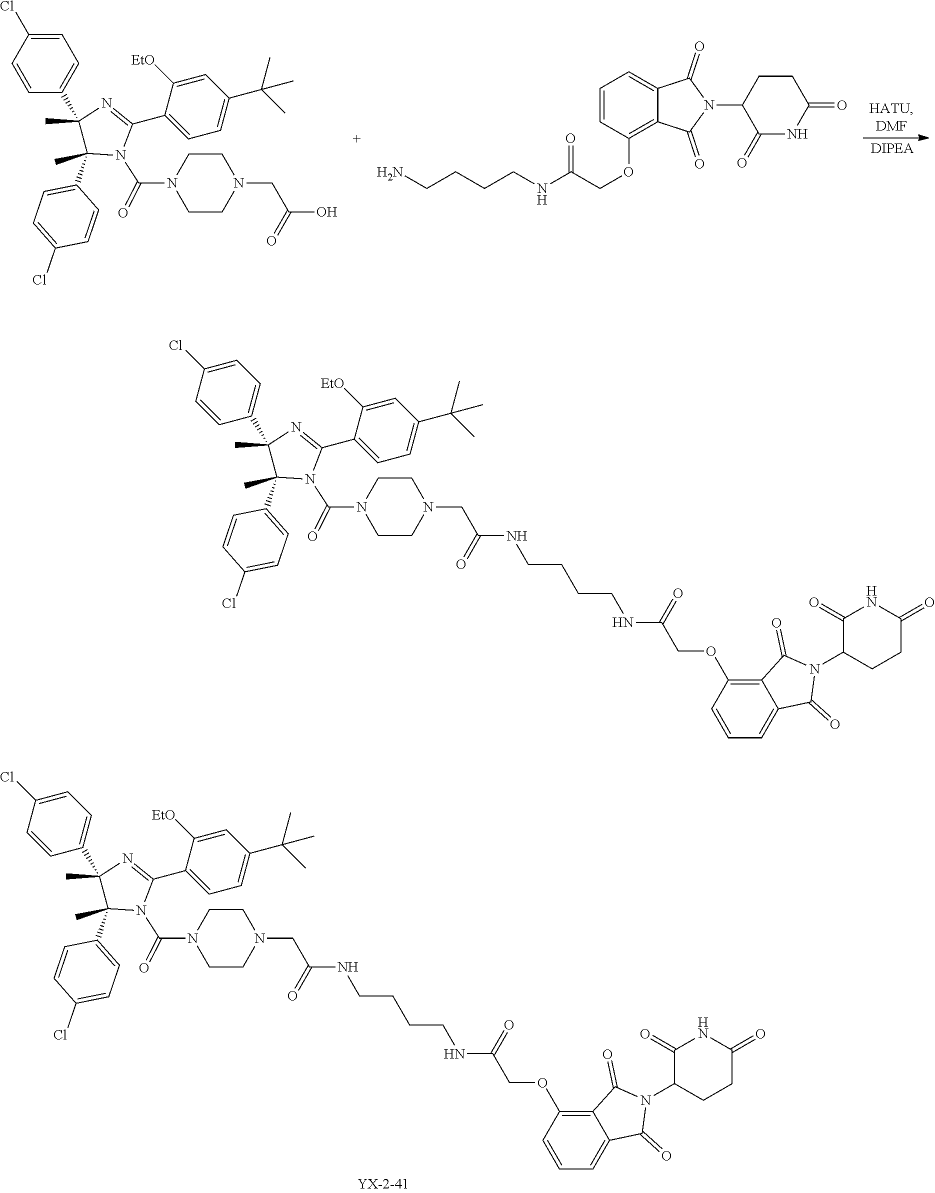 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1 ; NCCCCNC(=O)COc1cccc2c1C(=O)N(C1CCC(=O)NC1=O)C2=O ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCCCNC(=O)COc2cccc3c2C(=O)N(C2CCC(=O)NC2=O)C3=O)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCCCNC(=O)COc2cccc3c2C(=O)N(C2CCC(=O)NC2=O)C3=O)CC1]

2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)-N-(4-(2-((2-(2,6-dioxopiperidin-3-yl)-1,3-dioxoisoindolin-4-yl)oxy)acetamido)butyl)acetamide (YX-02-041): A mixture of 2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (YX-02-023) (36 mmg, 0.05 mmol) and Celebron-amine (20 mg, 0.05 mmol) in DMF (3 mL) was treated with DIPEA (16 μL, 0.1 mmol) and then HATU (20 mg, 0.05 mmol) and stirred at ambient temperature for 24 hour. After which time the reaction mixture was diluted with H₂O and extracted with EtOAc. The organic layer was dried by Na₂SO₄ and concentrated in vacuum. The resulting material was purified by chromatography on silica to afford YX-02-041 (12 mg, 23% yield). ¹H NMR (400 MHz, DMSO) δ 11.11 (s, 1H), 7.94 (d, J=5.7 Hz, 1H), 7.80 (t, J=7.9 Hz, 1H), 7.58 (d, J=7.7 Hz, 2H), 7.49 (d, J=7.3 Hz, 1H), 7.38 (d, J=8.4 Hz, 1H), 7.05 (dd, J=24.7, 15.7 Hz, 9H), 5.12 (dd, J=13.0, 5.4 Hz, 1H), 4.75 (s, 2H), 4.23 (s, 1H), 3.95 (s, 1H), 3.13 (s, 3H), 3.02 (s, 2H), 2.98-2.80 (m, 4H), 2.65-2.54 (m, 3H), 2.21 (s, 1H), 2.03 (d, J=11.7 Hz, 1H), 1.88 (s, 3H), 1.59 (s, 3H), 1.29 (t, J=6.7 Hz, 4H). LC-MS RT=2.17, [(M+2H)/2]=526.

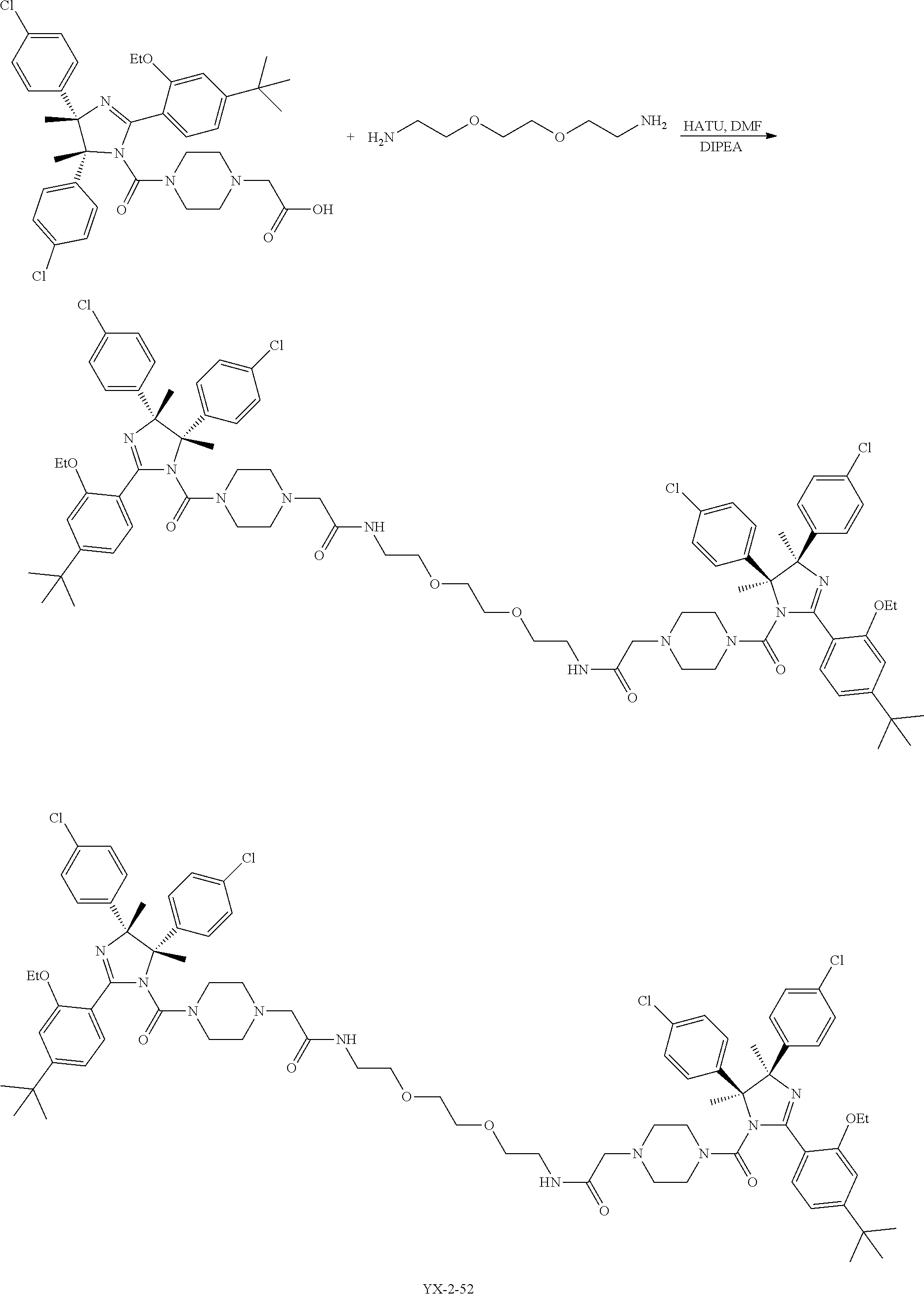

N,N′-((ethane-1,2-diylbis(oxy))bis(ethane-2,1-diyl))bis(2-(4-((4R,5S)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetamide) (YX-02-052): A mixture of 2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (36 mmg, 0.05 mmol) and 2,2′-(ethane-1,2-diylbis(oxy))diethanamine (YX-02-023) (YX-02-023 (3.7 μL, 0.025 mmol) in DMF (3 mL) was treated with DIPEA (16 μL, 0.1 mmol) and then HATU (20 mg, 0.05 mmol) and stirred at ambient temperature for 24 hour. After which time the reaction mixture was diluted with H₂O and extracted with EtOAc. The organic layer was dried by Na₂SO₄ and concentrated in vacuum. The resulting material was purified by chromatography on silica to afford YX-02-052 (30 mg, 84% yield). ¹H NMR (400 MHz, DMSO) δ 7.55 (dd, J=15.1, 7.1 Hz, 4H), 7.03 (d, J=31.7 Hz, 18H), 4.22 (s, 2H), 4.01-3.86 (m, 2H), 3.44 (s, 5H), 3.36 (t, J=5.8 Hz, 5H), 3.18 (d, J=5.1 Hz, 6H), 3.00-2.83 (m, 7H), 2.60 (s, 4H), 2.44 (d, J=10.1 Hz, 2H), 2.22 (d, J=9.4 Hz, 2H), 1.88 (s, 6H), 1.58 (s, 6H), 1.35 (s, 18H), 1.28 (t, J=6.8 Hz, 7H). LC-MS RT=2.09, [(M+2H)/2]=724.

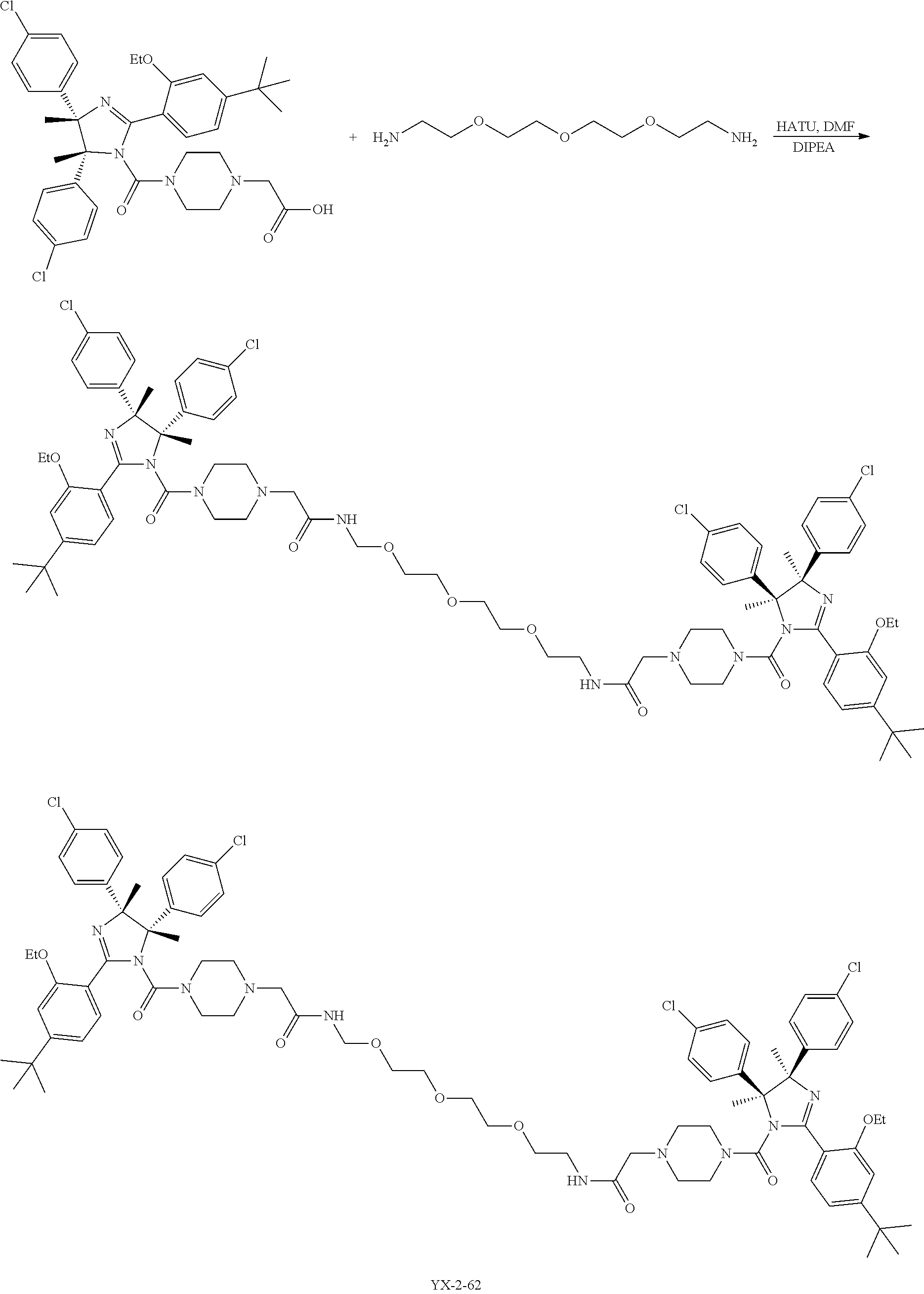

2-(4-((4R,5S)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)-N-(13-(4-((4R,5S)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)-12-oxo-2,5,8-trioxa-11-azatridecyl)acetamide (YX-02-062): A mixture of 2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (YX-02-023) (36 mmg, 0.05 mmol) and 2,2′-(2,2′-oxybis(ethane-2,1-diyl) bis(oxy))diethanamine (4.8 mg, 0.025 mmol) in DMF (3 mL) was treated with DIPEA (16 μL, 0.1 mmol) and then HATU (20 mg, 0.05 mmol) and stirred at ambient temperature for 24 hour. After which time the reaction mixture was diluted with H₂O and extracted with EtOAc. The organic layer was dried by Na₂SO₄ and concentrated in vacuum. The resulting material was purified by chromatography on silica to afford YX-02-062 (35 mg, 99% yield). ¹H NMR (400 MHz, DMSO) δ 7.95 (s, 1H), 7.57 (t, J=7.5 Hz, 4H), 7.13-6.92 (m, 18H), 4.22 (s, 2H), 4.00-3.89 (m, 2H), 3.46 (s, 7H), 3.37 (t, J=5.7 Hz, 4H), 3.18 (d, J=5.8 Hz, 6H), 2.92 (d, J=26.8 Hz, 9H), 2.73 (s, 3H), 2.69 (s, 2H), 2.58 (d, J=16.5 Hz, 4H), 2.44 (d, J=9.3 Hz, 2H), 2.22 (s, 2H), 1.88 (s, 6H), 1.59 (s, 6H), 1.36 (s, 17H), 1.29 (t, J=6.8 Hz, 7H). LC-MS RT=2.00, [(M+2H)/2]=745.

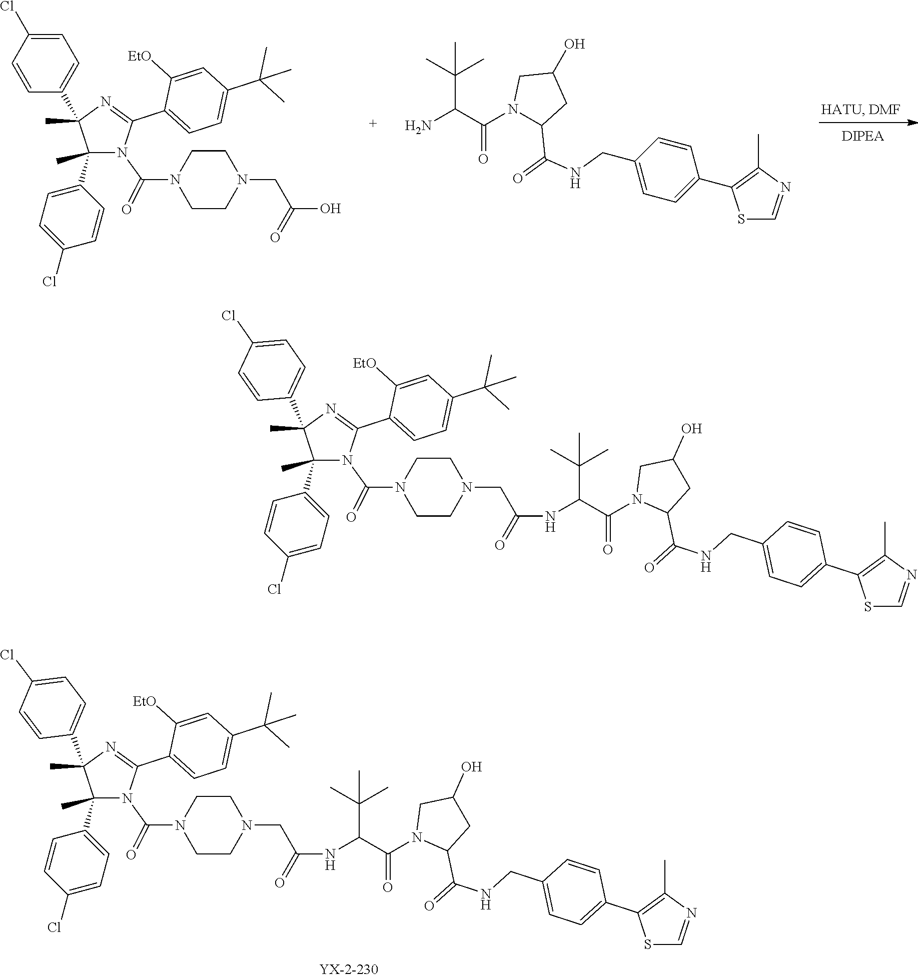 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1 ; Cc1ncsc1-c1ccc(CNC(=O)C2CC(O)CN2C(=O)C(N)C(C)(C)C)cc1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NC(C(=O)N2CC(O)CC2C(=O)NCc2ccc(-c3scnc3C)cc2)C(C)(C)C)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NC(C(=O)N2CC(O)CC2C(=O)NCc2ccc(-c3scnc3C)cc2)C(C)(C)C)CC1]

1-(2-(2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetamido)-3,3-dimethylbutanoyl)-4-hydroxy-N-(4-(4-methylthiazol-5-yl)benzyl)pyrrolidine-2-carboxamide (YX-02-230): A mixture of 2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (YX-02-023) (36 mmg, 0.05 mmol) and VHL-Ligand (25 mmg, 0.05 mmol) in DMF (3 mL) was treated with DIPEA (16 μL, 0.1 mmol) and then HATU (20 mg, 0.05 mmol) and stirred at ambient temperature for 24 hour. After which time the reaction mixture was diluted with H₂O and extracted with EtOAc. The organic layer was dried by Na₂SO₄ and concentrated in vacuum. The resulting material was purified by chromatography on silica to afford YX-02-230 (30 mg, 56% yield). ¹H NMR (400 MHz, DMSO) δ 8.99 (s, 1H), 8.56 (d, J=5.2 Hz, 1H), 7.57 (dd, J=12.0, 6.6 Hz, 2H), 7.48-7.33 (m, 4H), 7.03 (d, J=32.2 Hz, 9H), 5.11 (s, 1H), 4.48-4.36 (m, 2H), 4.33 (s, 2H), 4.31-4.15 (m, 2H), 3.95 (s, 1H), 3.60 (dd, J=30.0, 9.4 Hz, 2H), 3.16 (s, 1H), 2.90 (s, 3H), 2.78 (d, J=15.4 Hz, 1H), 2.65 (d, J=15.8 Hz, 1H), 2.44 (d, J=2.6 Hz, 4H), 2.30 (d, J=31.8 Hz, 2H), 2.10-1.97 (m, 1H), 1.97-1.81 (m, 4H), 1.59 (d, J=7.2 Hz, 3H), 1.35 (s, 8H), 1.28 (dd, J=16.0, 9.3 Hz, 4H), 1.02-0.73 (m, 11H). LC-MS RT=1.85, ES+ve 1079.

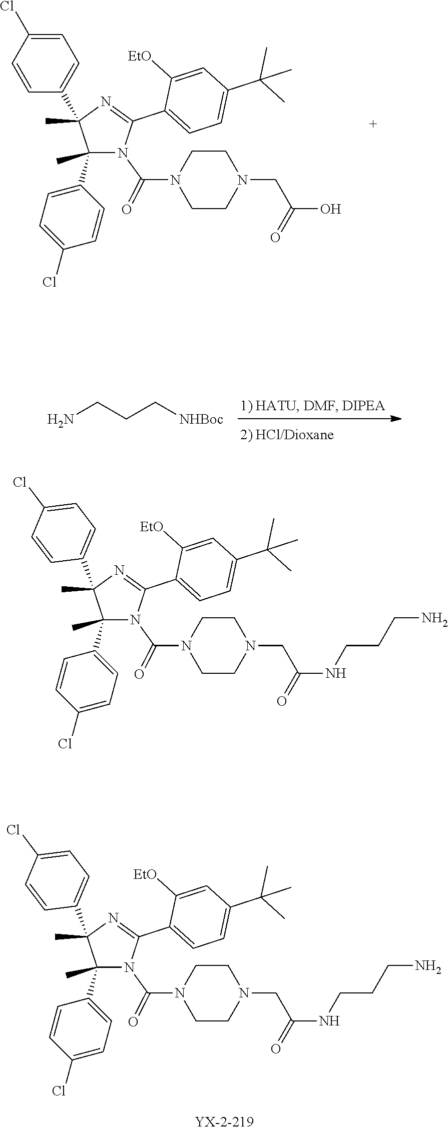 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1 ; CC(C)(C)OC(=O)NCCCN ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCCN)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCCN)CC1]

N-(3-aminopropyl)-2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetamide (YX-02-219): A mixture of 2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetic acid (60 mg, 0.083 mmol) (YX-02-023) and tert-butyl 3-aminopropylcarbamate (20 mg, 0.12 mmol) in DMF (4 mL) was treated with DIPEA (30 μL, 0.17 mmol) and then HATU (32 mg, 0.083 mmol) and stirred at ambient temperature for 24 hour. After which time the reaction mixture was diluted with H₂O and extracted with EtOAc. The organic layer was dried by Na₂SO₄ and concentrated in vacuum. The resulting material was purified by chromatography on silica to afford the coupling product YX-02-219 (55 mg, 80% yield). Then treat the product with Dioxane/HCl solution (4.0 M, 0.1 mL) and stir for 2 hours, remove the solvent and use directly for next step.

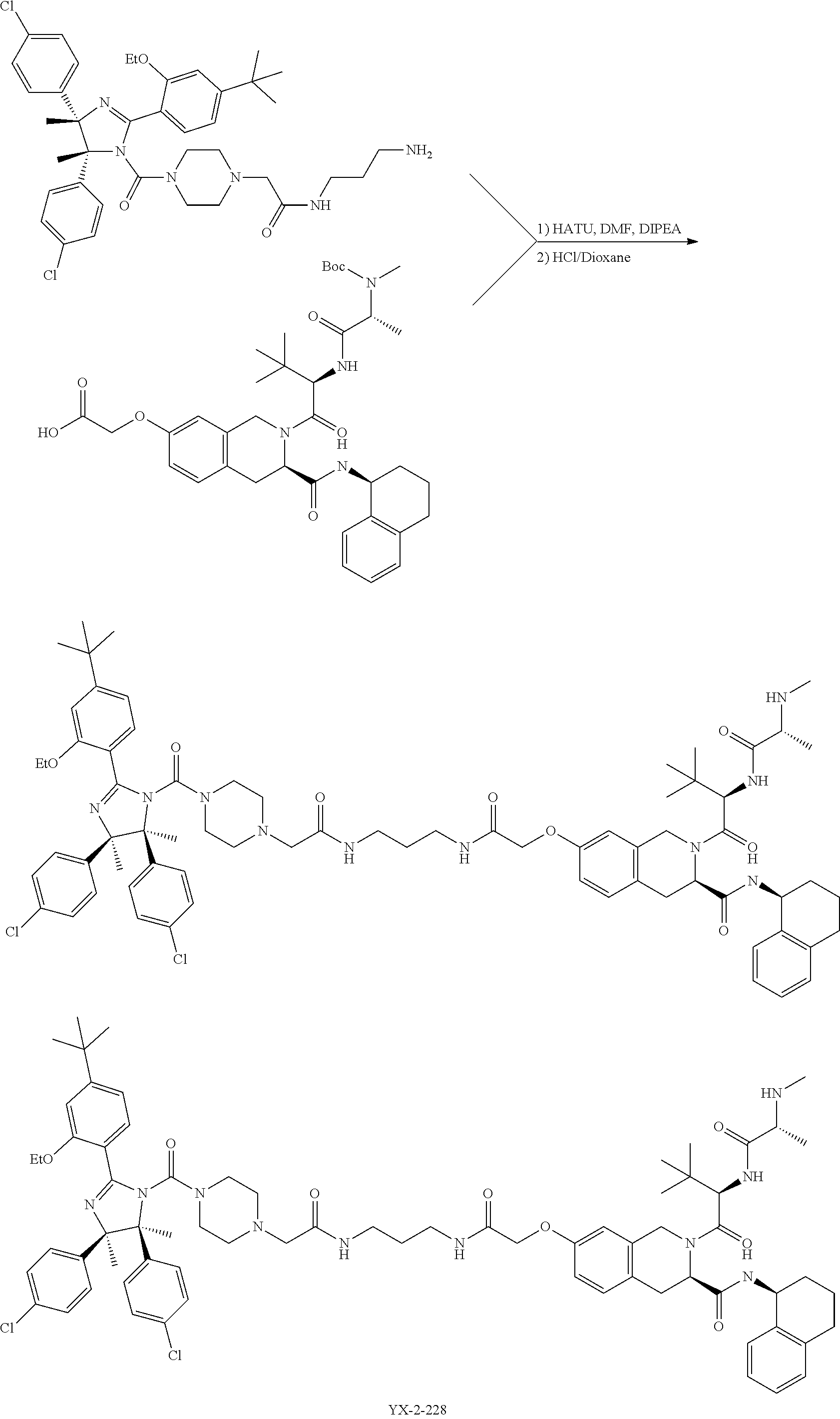

(R)-7-(2-((3-(2-(4-((4R,5S)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetamido)propyl)amino)-2-oxoethoxy)-2-((R)-3,3-dimethyl-2-((R)-2-(methylamino)propanamido)butanoyl)-N-((S)-1,2,3,4-tetrahydronaphthalen-1-yl)-1,2,3,4-tetrahydroisoquinoline-3-carboxamide (YX-02-228): A mixture of N-(3-aminopropyl)-2-(4-((4S,5R)-2-(4-(tert-butyl)-2-ethoxyphenyl)-4,5-bis(4-chlorophenyl)-4,5-dimethyl-4,5-dihydro-1H-imidazole-1-carbonyl)piperazin-1-yl)acetamide (YX-02-219) (18 mg, 0.025 mmol) and IAP-Ligand (20 mg, 0.025 mmol) in DMF (3 mL) was treated with DIPEA (20 μL, 0.05 mmol) and then HATU (12 mg, 0.025 mmol) and stirred at ambient temperature for 24 hour. After which time the reaction mixture was diluted with H₂O and extracted with EtOAc. The organic layer was dried by Na₂SO₄ and concentrated in vacuum. The resulting material was purified by chromatography on silica and then treated with Dioxane/HCl solution (4.0 M, 0.1 mL) for 2 hour, the reaction solvent was removed and purified by reverse-HPLC to afford YX-02-228 (10 mg, 31% yield). LC-MS RT=1.76, [(M+2H)/2]=642.

### Example 2—Analysis and Activity of Mdm2 Degrader

A. Mdm2 is Required for p53-Null T-Cell Lymphoma and Sarcoma Survival.

Previous work has shown that Mdm2 deletion inhibits growth and survival of both p53^{-/-} T-cell lymphoma and sarcoma by inducing apoptosis (Feeley et al., 2017). CreER, a 4-Hydroxytamoxifen (4-OHT) inducible form of Cre⁶¹ was expressed (retrovirus) in the lymphoma and sarcoma cells derived from Mdm2^{fl/fl}p53^{-/-} mice (Grier et al., 2002; Donehower et al., 1992). Loss of Mdm2 protein occurred with 4-OHT addition (FIG. 10A) resulting in reduced lymphoma cell viability (FIG. 10B), increased Annexin V positivity (FIG. 10C), and fragmented (sub-G1) DNA (FIG. 10D) (Feeley et al., 2017). Analogous results were obtained in the sarcoma cells (Feeley et al., 2017). These data indicate that Mdm2 deletion in either p53^{-/-} T-cell lymphoma or sarcoma inhibit their growth and survival by inducing apoptosis.

B. Mdm2 Deletion Induces Apoptosis of p53^{-/-} T-Cell Lymphoma and Sarcoma In Vivo.

To test whether Mdm2 deletion would impact p53-null tumor growth and survival in vivo, CreER expressing Mdm2^{fl/fl}p53^{-/-} T-cell lymphoma cells were injected subcutaneously into nude mice. Following detection of palpable tumors (day 15), tamoxifen (Tam) or corn oil (Oil) vehicle control was administered to all mice to activate CreER. Lymphomas in the vehicle control group continued to increase in volume, whereas the lymphomas in the mice given Tam to delete Mdm2 regressed and were undetectable at day 21 when the first control treated mouse was sacrificed (FIG. 11A). This reduced lymphoma burden resulted in a significant increase in survival compared to mice receiving vehicle control (FIG. 11B) (Feeley et al., 2017).

Analogous results were obtained in vivo with the sarcoma cells (Feeley et al., 2017). Evaluation of tumors harvested 48 hours after Tam or vehicle control administration showed loss of Mdm2, increased Annexin V positivity, and fragmented (sub-G1) DNA (FIG. 11C). Since tumors did ultimately grow in the mice receiving Tam, their Mdm2 levels were evaluated and observed that those that grew out expressed Mdm2 protein (Feeley et al., 2017), indicating the tumors formed from cancer cells that had not deleted Mdm2. Thus, Mdm2 loss in p53^{-/-} lymphoma or sarcoma in vivo caused apoptosis, which significantly diminished tumor growth resulting in prolonged survival of the mice.

C. Mdm2 Deletion Activates p53/p73 Transcriptional Target Genes which Induces Apoptosis in Murine p53-Null Cancer Cells.

RNA-sequencing was performed following Mdm2 deletion and compared this to vehicle control in the p53-null murine cancers, and this showed that Mdm2 deletion upregulated pro-apoptotic and cell cycle arrest genes known to be transcriptionally regulated by the p53 family (Feeley et al., 2017). p73, a p53 family member, can bind Mdm2 and is capable of mediating apoptosis and cell cycle arrest by transactivating p53 target genes (Ongkeko et al., 1999; Zeng et al., 1999; Wu & Leng, 2015). It was investigated whether p73 was involved in transcriptionally activating the p53 regulated genes and mediating the apoptosis observed. Mdm2 deletion increased p73 protein, and knockdown of p73 prevented the upregulation of the apoptotic target genes and the apoptosis induced by loss of Mdm2 in murine cancer cells lacking p53 (Feeley et al., 2017).

D. Mdm2 Knockdown in Human Cancer Cells with Mutated p53 Activates p73 and Induces Apoptosis

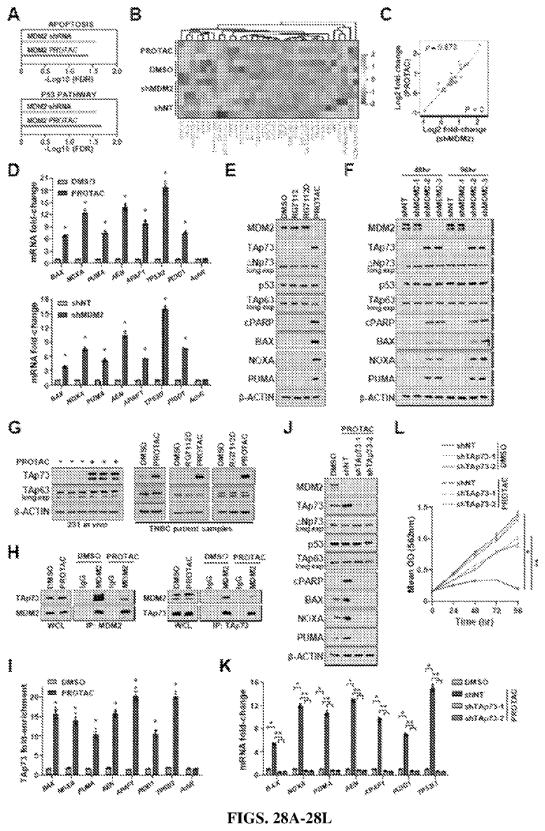

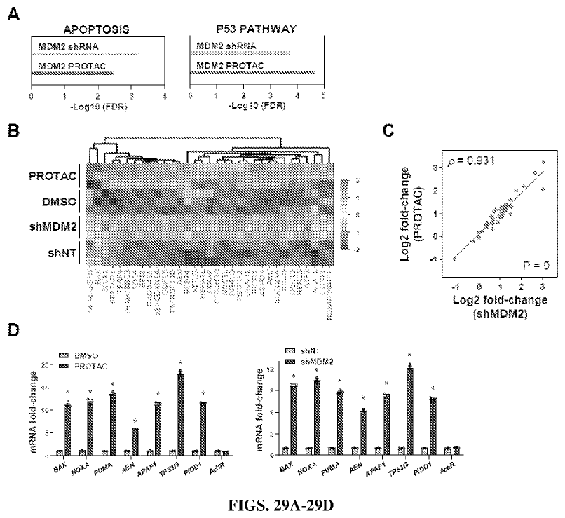

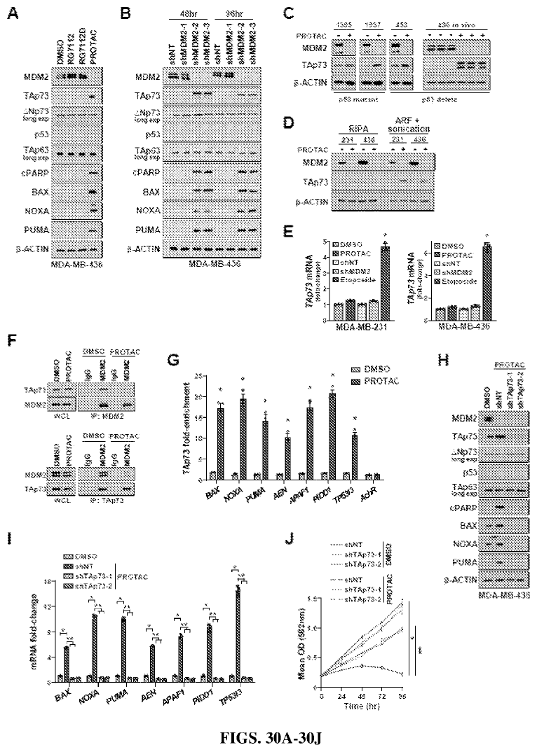

To determine whether the results in p53-null murine cancers translate to human cancers and in particular p53 mutant cancers, Mdm2 were knocked down with shRNAs in breast cancer cells with mutated p53. Mdm2 knockdown (FIGS. 28F and 30B) resulted in elevated TAp73 (isoform that is transcriptionally active) (FIG. 12A), increased BAX, PUMA, and NOXA mRNA expression (FIGS. 28D and 29D), decreased cell growth (FIG. 12B), and induced apoptosis (FIGS. 12A and 12C, cleaved PARP). FIG. 12D is RNA-seq comparing differential gene expression between MDM2 shRNA knockdown and YX-02-030. Analogous results were obtained in a breast cancer line that was p53-null. Together, the data indicate Mdm2 is required for the continued survival of both mouse and human cancer cells that have inactivated p53 either by deletion or mutation and suggest p73 plays a role.

### E. Development of Assays to Support Medicinal Chemistry Lead Optimization Efforts

i. Mdm2 Homogeneous Time-Resolved Fluorescence (HTRF) Binding Assays

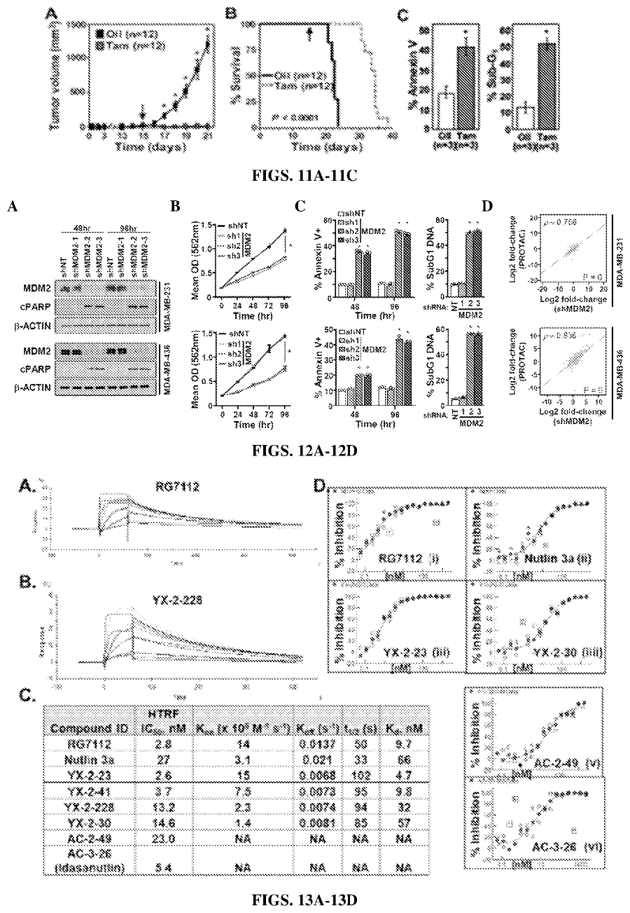

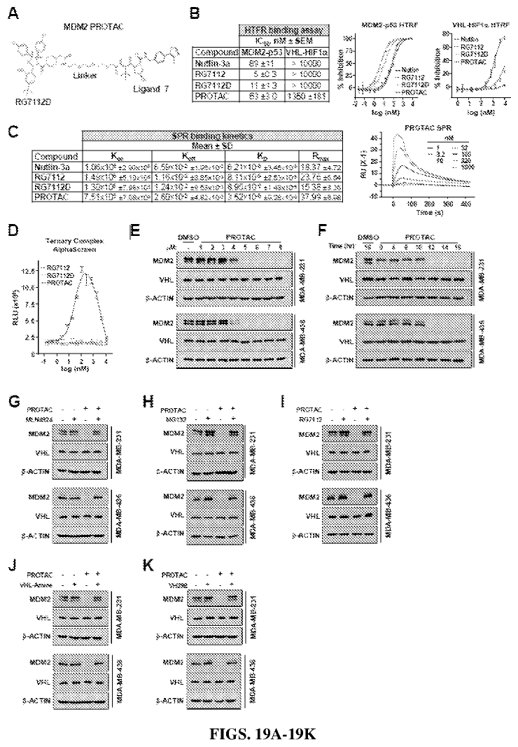

An HTRF binding assay was developed using a biotinylated peptide containing the Mdm2-binding domain of p53 and a recombinant GST-tagged N-terminus of Mdm2 containing the p53-binding domain. HTRF is a proximity-based fluorescence energy transfer (FRET) assay that takes advantage of the unique time-resolved fluorescence of the lanthanide chelates, such as terbium and europium. HTRF IC₅₀ values are provided for all key compounds (FIG. 13C, FIG. 19B, and Table 2).

ii. Mdm2 Surface Plasmon Resonance (SPR) Binding Assays.

SPR assays were developed to measure the direct label free binding to Mdm2. Assay optimization involved protein coupling to the sensor chip through non-specific amine coupling and directed coupling using a His tagged protein. The best results were obtained using the direct coupling method resulting in approximately 10,000 RU of His-SUMO-Mdm2 bound to the sensor chip. Control Mdm2-p53 inhibitors RG7112 (Kd=9.7 nM; FIG. 13C and FIG. 19C) and Nutlin3a (Kd=66 nM; FIG. 13C and FIG. 19C) were then tested. Senosgrams for RG7112 (FIG. 13A) and PROTAC YX-02-228 (FIG. 13B) are shown. The SPR data generally correlates with the Mdm2-p53 HTRF binding assay (FIG. 13C). The differences in the affinity of the bi-functional compounds, YX-02-228, YX-02-030, and YX-02-041, compared to the parent compound is driven by association rate. The dissociation rates for inhibitors versus bi-functional compounds were found to be similar. These data confirm high affinity binding to Mdm2 with potential PROTACs.

iii. HTRF Binding Assays for Cereblon, VHL, and cCbl.

Biochemical assays in 384-well format have established to assess the binding of newly synthesized PROTACs to the appropriate E3 ligase using HTRF technology. In these assays an anti-HIS antibody conjugated to terbium was used as the donor fluor, and either a Cy5 labeled small molecule probe, or a biotinylated peptide that is bound to a streptavidin acceptor. See TABLE 2.

TABLE 2
HTRF Binding Data for PROTACs

| | Mdm2 | VHL | CRBN | |
|---|---|---|---|---|
| | HTRF | Binding | Binding | Ligand |
| ID | IC₅₀ nM | IC₅₀ nM | IC₅₀ nM | series |
| AC-3-049 | 23 | [symbol] | >10 uM | Idasanutlin |
| YX-2-228 | 13 | >10 uM | >10 uM | RG7112 |
| YX 2-230 | 18 | [symbol] | >10 uM | RG7112 |
| YX-2-30 | 14 | [symbol] | >10 uM | RG7112 |
| YX 2-41 | 3 | >10 uM | [symbol] | RG7112 |

[symbol]

indicates data missing or illegible when filed

cCBL: Recombinant human cCBL protein with an N-terminal HIS tag was combined with anti-HIS-Tb Cryptate Gold (CisBio) in low volume 384-well plates. Test compounds are added, then biotinylated phospho-tyrosine containing peptide (IK(-Biotin)NTNEpYTEGPTV (SEQ ID NO: 1)). After incubation the HTRF signal is measured on the Envision. Data is normalized to % inhibition, where the baseline HTRF signal is determined in the absence of cCBL, but including all other assay components, and is defined as 100%. The total HTRF signal in the absence of test compound is defined as 0%. The typical signal-to-noise is 5:1. Potent “hit” ligands have been identified and will be evaluated as new E3 recruiting ligands.

Cereblon: Recombinant human cereblon (CRBN) complex, including CUL4A, RBX1, DDB1 and CRBN is purchased from R & D systems. This four-member protein complex has an N-terminal HIS tag on the CUL4A subunit. The CRBN complex is diluted in assay buffer containing anti-HIS-Tb Cryptate Gold (CisBio) and is added to low volume 384-well plates. Test compounds in 100% DMSO are added to the plates followed by the Cy5-labeled lenolidamide probe. After 1 hour the HTRF signal is measured on the Envision. Data are normalized to % inhibition. The typical signal-to-noise is 5:1. Bi-functional molecules containing the Cereblon ligands are tested in TABLE 2 (see YX-02-041).

### F. ICW Assays to Screen for Protein Degradation.

i. Linearity Protocol

Cells were seeded at decreasing cell number from 15K cells to 2K cells. The Mdm2 primary antibody was tested at 1:200 and 1:300 dilution. The secondary antibody conjugated to IRDye800CW was used with 1:800 dilution, and CellTag 700 Stain was used at 1:1000 dilution as manufacture recommended. The linearity of cell number versus signal was generated with Prism. The signal over background ratio was also calculated. The seeding cell number and primary antibody dilution factor was determined based on these data.

ii. ICW Protocol

Cells are seeded in 96 half area well plates (black clear bottom) and cultured overnight. Then they are treated with compounds, fixed with 4% paraformaldehyde for 20 minutes, and permeabilized in 0.1% Triton. They are then treated with the primary antibody and secondary antibody conjugated to IRDye800CW or treated with CellTag 700 Stain. The plates are then scanned on the OdysseyCLX and the data processed to provide a dose response curve.

### G. Design and Chemical Synthesis of Mdm2 Targeting PROTACs

Mdm2 targeting ligands were utilized that have advanced into human clinical trials since these have undergone a significant level of optimization, particularly for off-target selectivity and in vivo pharmacokinetic exposure. RG7112, developed by Hoffman LaRoche, is the first Mdm2 inhibitor to enter clinical trials (Ray-Coquard et al., 2012). RG7112 is an improved derivative over its precursor Nutlin-3a (Vassilev et al., 2004). AMG-232 is a potent sub-nanomolar piperidinone Mdm2 targeting ligand developed by Amgen, with excellent reported pharmacokinetic properties (Rew & Sun, 2014). RG7388 (idasanutlin) is a second-generation derivative of RG7112 developed by Hoffman LaRoche. Idasanutlin is reported to have improved potency and selectivity over RG7112 (Ding et al., 2013). It is also the first Mdm2 inhibitor to move into Phase 3 clinical trials (Mdm2 ligands shown in FIG. 14A). The Mdm2 ligands were developed using rational structure-based design and FIG. 14B-D shows the X-ray structures of RG7112, p53, and p73 in complex with Mdm2 (all binding in the same Leu pocket) (Rew & Sun, 2014; Nag et al., 2014). The key binding interaction is where the p53 transactivation domain peptide interacts with Mdm2 (N-terminus region AA residues 1-109). The three key amino acids in p53 (Phe19, Trp23 and Leu26) are essential for binding. RG7112, AMG-232, and Idasanutlin all contain a chemical functional group for linker attachment, such as an amine or carboxylic acid (shown in Orange; FIG. 14A). The position in Mdm2 where the recruiting ligands bind in the ternary complex is the same site where p53 and p73 bind (FIG. 14B-6D).

### H. In-Cell Western Assay to Confirm Degradation.

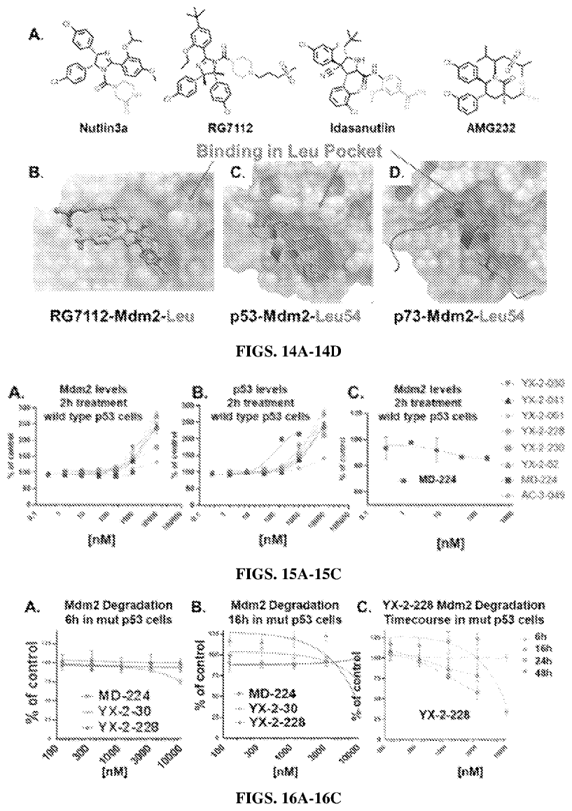

The potential PROTACs were evaluated in the wt p53 MCF7 cell line and in the mut p53 MDA-MB-231 cell line. Importantly these were compared to MD-224, which was recently reported to potently degrade Mdm2 in RS4; 11 leukemia cells (Li et al., 2019). Assay linearity was performed confirming a strong 4-fold signal to noise in the assay. A series of bi-functional molecules were then treated and evaluated for protein level in the assay over time from 2 h-48 h. In FIG. 15A, most compounds showed significantly increased Mdm2 levels. In this wt p53 cancer cell line the stabilization of p53 (FIG. 15B), and subsequent p53-mediated transcriptional upregulation of Mdm2 make it difficult to observe degradation. In FIG. 15C, it was shown that the reference compound MD-224 shows modest degradation. These compounds were evaluated in the mutant p53 cancer cells. In FIG. 16A-16C, YX-02-030 and YX-02-228 were shown to degrade Mdm2 in mutant p53 MDA-MB-231 cancer cells. In this cell line, it takes approximately 16-24 h to detect the degradation. Surprisingly, the reported Mdm2 degrader, MD-224, did not reduce Mdm2 protein level (FIG. 16A-B), showing no effect or slightly increased protein levels (even over a time course of 2 h-48 h). YX-02-030 and YX-02-228 utilize highly selective clinically used Mdm2 targeting ligands, in the same manner as MD-224 however, these PROTACs use VHL and cIAP E3 ligase recruiting ligands, respectively. The difference in the molecules is that MD-224 utilizes a CRBN recruiting ligand. The compound, YX-02-041, which also uses a CRBN recruiting ligand (TABLE 1) did not provide robust degradation in the mutant p53 cancer cells. Without wishing to be bound by any theory, it is believed that CRBN is not a preferred E3 in this situation and further supports the use of alternate E3 ligase recruiting ligands during PROTAC optimization.

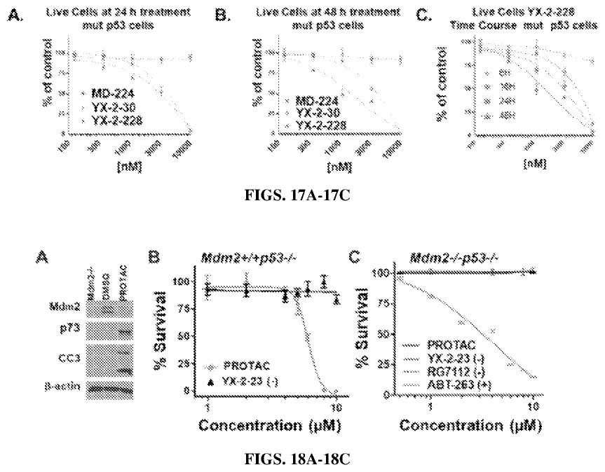

Since genetic deletion of Mdm2 induces p73 and induces apoptosis in p53 mutant and deficient cancer cells, YX-02-030 and YX-02-228 were evaluated for their effect on cell viability. First, the CellTag700 Stain was utilized to count the number of live cells in the ICW format. Interestingly, YX-02-030 and YX-02-228 show potent cytotoxic effects. YX-02-030 and YX-02-228 reduced the number of live mut p53 MDA-MD-231 cells remaining after 24 h and 48 h treatment, whereas MD-224 had no cytotoxic effect (FIG. 17A-B). Notably, in other studies, the compound YX-02-041, which uses the CRBN recruiting ligand (like MD-224) was cytotoxic to the wild type p53 cells (MCF7; IC₅₀-20 μM) but much less cytotoxic nor did it degrade mutant p53 cells. Therefore, without wishing to be bound by any theory, it is believed that CRBN is not optimal for degrading mutant p53 cells. YX-02-228 treatment showed similar results (shown over 6-48 hour time course; FIG. 17C). The degrading ability and cell growth inhibitory action of YX-02-030 was confirmed by traditional Western blot analysis and MTT assay in a small panel of human carcinoma cells with varying p53 status (FIG. 21F) to validate the findings in the In-Cell Western assay (See, FIG. 19E and FIG. 19F for validation of the in cell westerns in MBA-MD-231 cells and MDA-MB-436 cells. FIG. 18A shows loss of Mdm2 in mouse sarcoma cells with YX-2-30). Notably, VHL is not degraded by YX-02-030, possibly due to modest VHL binding also, non-cancerous cells were resistant to YX-02-030. YX-02-030 was further evaluated in comparison to YX-02-023 (the RG7112 derivative) using the p53-null mouse sarcoma cells for cell survival, activation of p73, and Mdm2 degradation analysis (FIG. 18). YX-02-030 treatment stabilized p73 and induced apoptosis (appearance of cleaved caspase 3) with Mdm2 degradation (FIG. 18A), mimicking what is observed with genetic deletion of Mdm2 (Feeley et al., 2017). Also, increasing concentrations of YX-02-030, but not the controls YX-02-023 (RG7112 derivative) or DMSO, killed p53-null sarcoma cells (FIG. 18B). Mdm2 must be present initially for the YX-02-030 PROTAC to have a cytotoxic effect (FIG. 18C), demonstrating YX-02-030 is not acting like a molecular glue (i.e. the E3 recruiting ligand acting independently of the targeting ligand). Note that the Mdm2^{-/-}p53^{-/-} sarcoma cells are not responsive to YX-02-030, but are responsive to other compounds (e.g., the Bcl-2/X/W inhibitor ABT-263/Navitoclax). YX-02-030 was then tested to determine if the compound had any effect in the absence of p73, to further confirm the hypothesis that p73 was mainly responsible for the loss of viability effects that was observed. Using 2 different shRNAs to knockdown p73; mut p53 MDA-MB-231 cancer cells were protected from YX-02-030 induced cell death. See, the data in FIGS. 28J, 28L, 30H, and 30J. These data strongly support the hypothesis that Mdm2 degradation via Mdm2-targeted PROTACs kills cancers with inactivated p53 by activating the p53-independent activities of Mdm2.

### I. YX-02-030 Targets Mdm2 for 26S Proteasome Degradation Via VHL-Dependent Ubiquitination.

Treatment with the proteasome inhibitor MG132 prevented YX-02-030 mediated Mdm2 degradation (FIG. 19H). Addition of excess RG7112 to cells also prevented YX-02-030-mediated Mdm2 degradation (FIG. 19I). Analogously, excess VHL recruiting ligand prevented YX-02-030-mediated Mdm2 degradation (FIG. 19J), and notably while Mdm2 is an E3 ligase, there was no change in VHL suggesting that YX-02-030 did not target VHL (FIG. 19E-19J). Similar results were obtained with a VHL small molecule inhibitor, VH298 (FIG. 19K). These results strongly suggest that degradation by YX-02-030 requires the formation of the ternary complex between Mdm2, YX-02-030, and VHL to initiate VHL-dependent ubiquitination and Mdm2 proteasomal degradation in these p53 deficient or mutant cancer cells.

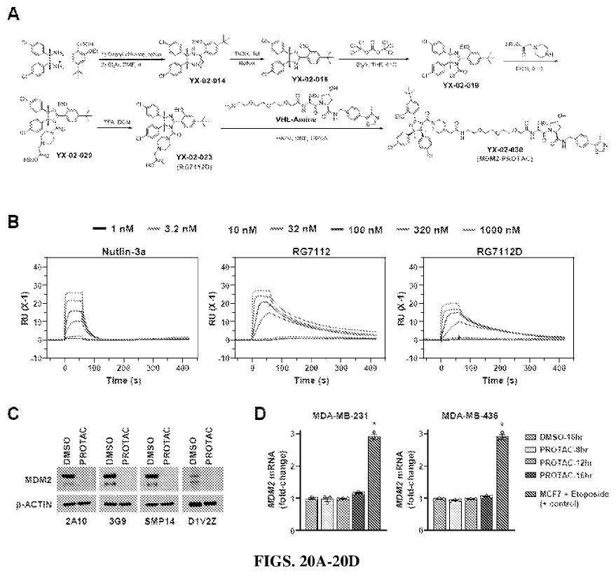

PROTACs work by forming a ternary complex consisting of the target protein, the PROTAC, and the recruited E3 ubiquitin ligase-E2 complex, causing ubiquitination of the target protein, marking it for 26S proteasome-mediated degradation (Nalawansha & Crews, 2020). FIG. 19D confirms that the PROTAC forms a tertiary complex. The MDM2-directed PROTAC (YX-02-030) showed a concentration (FIG. 19E) and time-dependent (FIG. 19F) loss of MDM2 protein in TNBC cells with either mutated p53 (MDA-MB-231) or deleted p53 (MDA-MB-436). Since MDM2 can be phosphorylated, which can block antibody binding sites (Eischen, 2011), multiple MDM2 antibodies were tested and all showed loss of MDM2 protein with PROTAC exposure (FIG. 20C). Moreover, loss of MDM2 protein was not due to decreased MDM2 mRNA levels, as they remained unchanged with PROTAC treatment (FIG. 20D). Testing the contribution of the ubiquitin cascade with MLN4924, a NEDD8-activating enzyme inhibitor critical for ubiquitin transfer (Soucy et al., 2009), showed it antagonized PROTAC-mediated MDM2 protein degradation in TNBC cells (FIG. 19G). Similarly, when the proteasome was inhibited with MG132, MDM2 protein levels were maintained despite YX-02-030 exposure (FIG. 19H). Therefore, YX-02-030 targets MDM2 protein for 26S proteasome-mediated degradation in TNBC cells and this requires the ubiquitin cascade.

To determine whether the ternary complex was required for MDM2 degradation, competition for binding to MDM2 and VHL was carried out using adding excess RG7112 or VHL-Amine, respectively. PROTAC-induced MDM2 degradation was prevented with either RG7112 or VHL-Amine (FIGS. 191 and 19J, respectively). Analogous results were obtained using VH298, a VHL-specific small molecule inhibitor ((Frost et al., 2016); FIG. 21K). Thus, ternary complex formation of MDM2, YX-02-030, and VHL is required for PROTAC-induced MDM2 degradation.

J. Reduced p53-Inactivated TNBC Clonogenic Potential and Increased Mammosphere Apoptosis with MDM2-PROTAC Treatment

The ability of the PROTAC-mediated degradation of MDM2 to alter the clonogenic potential of p53-inactivated TNBC cells was evaluated by performing colony formation assays with MDA-MB-231 and MDA-MB-436 cells. Following YX-02-030 treatment, there was a significant reduction in colony numbers with 1 M of YX-02-030 (FIG. 21A). There was little/no effect with 2 M of MDM2 inhibitors, but <2 colonies formed with 2 M of YX-02-030 (FIG. 21A).

These were then evaluated in 3D culture as to when the YX-02-030 was effective at preventing TNBC cell mammosphere formation and eliminating already formed mammospheres. For both MDA-MB-231 and MDA-MB-436 cells, significantly fewer mammospheres formed at each YX-02-030 concentration tested, compared to the MDM2 inhibitors and vehicle control (FIG. 21B). In already-established mammospheres, there was a significant decrease in mammosphere area following YX-02-030 treatment (FIG. 21C). Notably, a 3-fold lower concentration of YX-02-030 than the MDM2 inhibitors reduced mammosphere area by 49.5% and 43.8% in MDA-MB-231 and MDA-MB-436 mammospheres, respectively, and equal concentrations reduced mammosphere area even more (FIG. 21C). This reduction was due to apoptotic cell death, as we detected significantly increasing Caspase-3/7 activity over time (FIG. 21D) and decreased survival (FIG. 21E) in the mammospheres following YX-02-030 treatment compared to the MDM2 inhibitors. Together, these data provides strong evidence that YX-02-030 induces apoptosis in p53-inactivated TNBC cells in both 2D and 3D culture models.

### K. Mouse Liver Microsomal Stability and In Vivo Pharmacokinetic Exposures of Mdm2 Compounds.

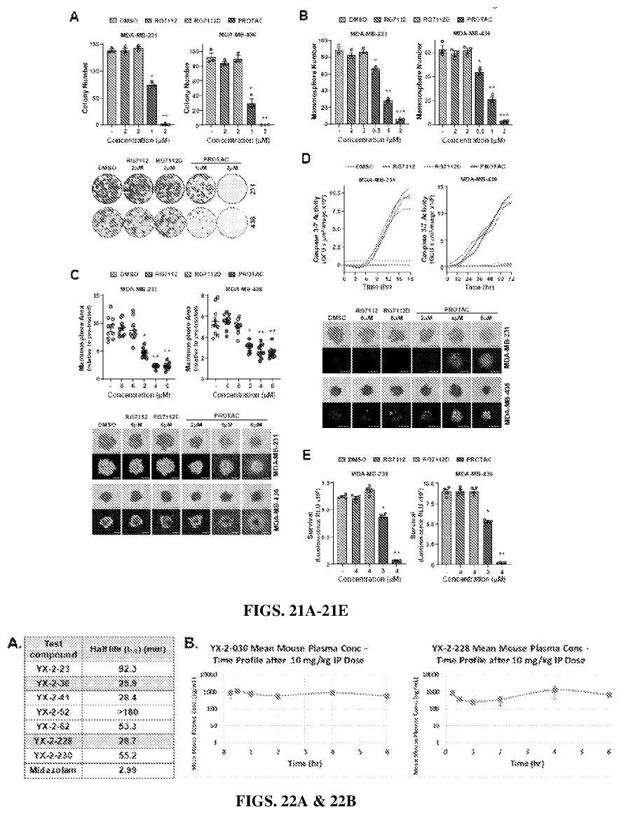

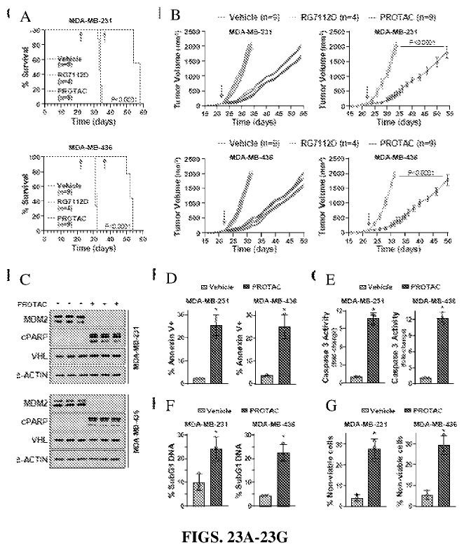

To translate cell-based activity to in vivo efficacy, the Mdm2 PROTACs were evaluated for metabolic stability and plasma compound levels after IP injection in the mouse. Mouse liver microsome stability was obtained for lead compounds (Alliance Pharma, Inc; Malvern PA) as a predictive measure for suitable exposure in a mouse pharmacokinetic study. In brief, test compounds (0.5 μM) are incubated with liver microsomes (0.5 μg/mL) and an NADPH-regenerating system (cofactor solution) and samples are taken at various timepoints, quenched with an acetonitrile solution containing an internal standard, then analyzed by LC-MS/MS. These data provide half-life of parent remaining and intrinsic clearance (CLint) determined from the first-order elimination constant by nonlinear regression (FIG. 22A). Mouse pharmacokinetic studies for YX-02-030 and YX-02-288 were completed. Mouse plasma levels were evaluated following intraperitoneal (10 mg/kg) administration. Plasma was collected at timepoints 0.25 h, 0.5 h, 1 h, 2 h, 4 h and 6 h after Mdm2 PROTAC injection. Plasma was deproteinized and analyzed for compound using LC-MS/MS methods. Three mice were used for each time point and one control animal (Alliance Pharma Inc, Malvern PA). Both YX-02-030 and YX-02-228 had prolonged in vivo plasma exposure (FIG. 22B). Together, the data show that the Mdm2 PROTACs YX-02-030 and YX-02-228 are cell permeable, are active in cell-based assays (as shown by degradation and effects on cell viability) and possess suitable physiochemical properties predictive of reasonable pharmacokinetic exposure in vivo for translation to in vivo efficacy studies. Thus, although YX-02-030 and YX-02-228 are larger than typical small molecule drugs they possess suitable attributes for in vivo translation into mouse studies by IP administration.

L. In Vivo Efficacy Study with Mdm2 PROTAC, YX-02-030.

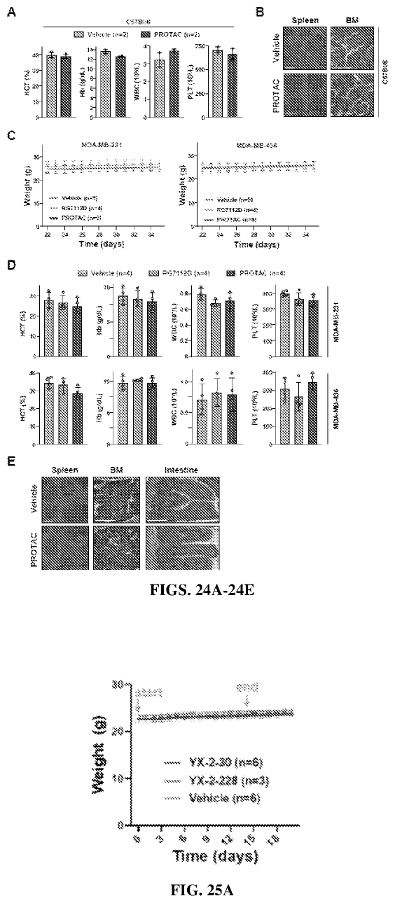

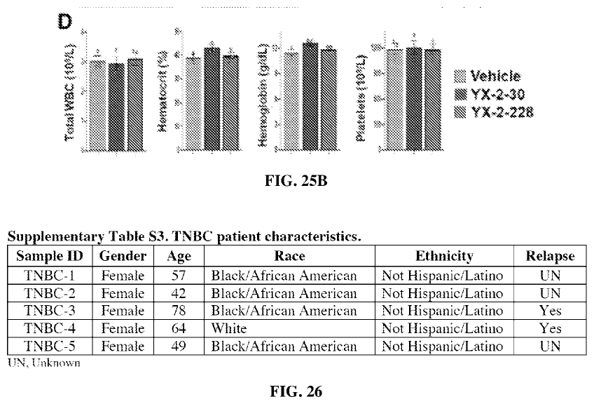

Prior to testing YX-02-030 tumors in vivo, mouse liver microsome and pharmacokinetic (PK) studies were performed to determine its stability in vivo. YX-02-030 was moderately metabolically stable with a 25.9-minute half-life (FIG. 22A) and had excellent in vivo stability with plasma levels stable over 6 hours in mice after a single 10 mg/kg intraperitoneal dose (FIG. 22B). Due to its stability in vivo, the effectiveness of YX-02-030 was then evaluated at killing TNBC tumors in mice. Xenografts of MDA-MB-231 and MDA-MB-436 TNBC cells grew to ˜80 mm³, and then tumor size-matched mice were treated with YX-02-030, RG7112D control compound, or vehicle control. Fourteen days of MDM2-PROTAC treatment significantly extended mouse survival (FIG. 23C) and decreased tumor volume (FIG. 23D), compared to control mice for both xenograft models. To confirm YX-02-030 was hitting its target (MDM2) in the TNBC tumors, tumors were harvested from a cohort of mice after 72 hours of YX-02-030 treatment. Mice that received YX-02-030 showed loss of MDM2 protein (FIG. 23E) and increased cleaved PARP (FIG. 23E), Annexin-V positivity (FIG. 23F), Caspase-3 activity (FIG. 23G), subG1 apoptotic DNA (FIG. 23H), and non-viable cells (FIG. 23I). Notably, no signs of overt toxicity in immune-competent C57Bl/6 mice (FIG. 24A, B) or immune-deficient mice from the xenograft experiments (FIG. 24C-E) were observed following MDM2-PROTAC treatment. Specifically, mouse weight was maintained (FIG. 24C) and complete blood counts (FIGS. 24A & 24D) and the histology and cellular content of the spleen, bone marrow, and intestine were normal (FIGS. 24B & 24E). The toxicity of YX-2-228 was analyzed as compared to both the vehicle and YX-2-30. (FIGS. 25A & 25B). Therefore, YX-02-030 showed clear in vivo efficacy against p53-inactivated TNBC tumors and no obvious toxicity to normal tissues.

### M. The Use of Proteomics to Evaluate Protein Degradation

A proteomics analysis was performed to assess the selectivity of degradation by YX-02-030. MDA-MB-231 cells were treated for 8 hours with 5 μM of YX-02-030 or DMSO in triplicate and processed to provide 25 μg of protein loaded on a 10% acrylamide gel. The entire protein-containing gel regions were excised, digested with trypsin and analyzed by liquid chromatography tandem mass spectrometry (LC-MS/MS) using a 240-min gradient as described (Chae et al., 2016). Peptide sequences were identified using MaxQuant 1.6.3.3 (Cox & Mann, 2008). MS/MS spectra were searched against a UniProt human protein database. A total of 4237 significantly different protein groups were quantified. Under these experimental conditions Mdm2 was not able to be detected either in the treated or untreated samples.

However, this experiment demonstrates that protein degradation induced by YX-02-030 only affected <10% of the observable proteins. Only 31 proteins out of 4237 proteins were significantly changed, 4 changed greater than 100-fold, and 11 proteins were increased, and 19 proteins were decreased. Without wishing to be bound by any theory, it is believed that as Mdm2 is an E3 ubiquitin ligase, the decreases of these protein could be impacted and these would be detected in these methods. This experiment confirms the specificity of YX-02-030, and that it does not affect hundreds of proteins in a non-specific manner.

### N. YX-02-030 and YX-02-228 Treatment is Well Tolerated

In vivo tolerability tests were conducted with the 2 lead Mdm2 PROTACs to evaluate tolerated doses and effects on normal tissues. Nude mice were treated with Mdm2 PROTACs, YX-02-030 (50 mg/kg; IP) and YX-02-228 (30 mg/kg; IP) once daily for 8 days. Results showed no adverse clinical observations for spleen and bone marrow (FIGS. 24B & 24E), and there was no significant change in body weight (FIG. 25A). Blood counts were also evaluated and they show no significant change (FIG. 25B). YX-02-030 was also well tolerated in C57Bl/6 mice (30 mg/kg; IP; 2× daily) (See FIG. 24). These data suggest that the compounds are well tolerated and there are no acute toxicities observed with Mdm2 PROTAC treatment.

### O. Targeted MDM2 Degradation Induces Apoptosis in Patient-Derived TNBC Explants

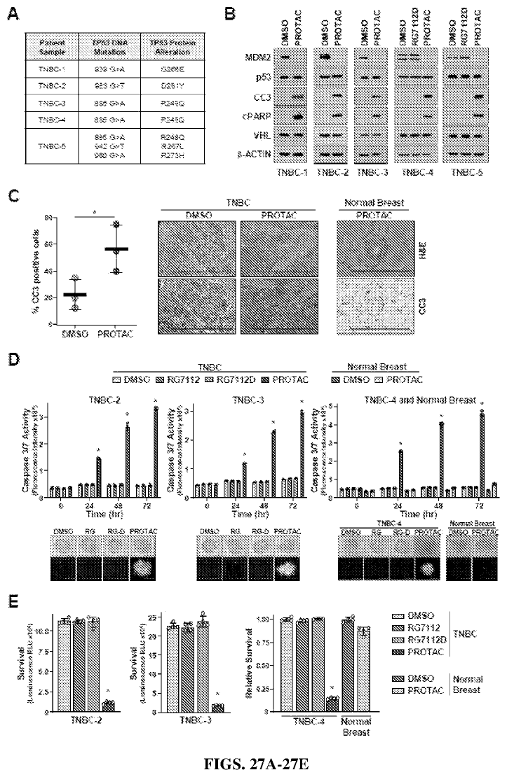

To assess the effects of YX-02-030 on TNBC patient samples, fresh, surgically-resected tumor from five TNBC patients was obtained, and 80% were from Black/African American patients (FIG. 26). Each tumor had TP53 missense mutations with two having the same hot-spot (R248Q) p53 mutation and one that had 3 TP53 mutations (FIG. 27A). While preserving tumor architecture, pieces of each patients' tumor were placed into explant cultures (Schiewer et al., 2012; Centenera et al., 2018) and subjected to treatment with YX-02-030 and vehicle control, and when enough tissue was provided, also RG7112D. Within 4 days of treatment, MDM2 protein was lost in the patient tumors that received the YX-02-030 compared to vehicle and RG7112D controls, and as expected, no change in VHL protein (FIG. 27B). YX-02-030-treated patient tumors were undergoing apoptosis, as those explants showed increased levels of cleaved Caspase-3 and cleaved PARP by Western blot (FIG. 27B). Immunohistochemistry also showed a significantly increased number of cleaved Caspase-3 positive tumor cells in MDM2-PROTAC-treated explants, but normal breast epithelial and stromal cells were unaffected (FIG. 27C). Thus, the data from the patient-derived TNBC explants show that YX-02-030 effectively and specifically kills TNBC patient tumor cells.

For three of the patient TNBC tumor samples there was enough tissue to also establish 3D mammosphere cultures, and for one of these patients, normal breast epithelial tissue was also received that generated loose cell clusters. YX-02-030 significantly increased Caspase-3/7 activity (FIG. 27D) and reduced survival (FIG. 29E) of the mammospheres from all three patient tumors, whereas MDM2 inhibitors had no effect. The normal breast epithelial clusters remained largely unaffected by YX-02-030 treatment (FIG. 27D, E). These data provide significant evidence that targeting MDM2 for degradation may be a viable, non-toxic therapeutic strategy for TNBC.

### P. TAp73 is Induced and Required for Apoptosis Upon MDM2-Targeted Loss in p53-Inactivated TNBC Cells

To gain insight into the mechanism by which YX-02-030 was killing p53-mutant and deleted TNBC cells, RNA-sequencing data were evaluated on cells treated with YX-02-030 and cells expressing MDM2 shRNA and each of their controls. As expected, evaluation of gene signatures from the Hallmark database (Liberzon et al., 2015) that were significantly enriched showed genes linked to apoptosis in both cell lines whether they were treated with YX-02-030 or MDM2 was knocked down (>2 fold-change, FDR<0.05; FIG. 28A; FIG. 29A). Additionally, our analysis also revealed p53 pathway gene signatures were also significantly enriched (>2 fold-change, FDR<0.05; FIG. 28A; FIG. 29A). More than 25 genes targeted by the p53 family, as reported in the IARC TP53 Database (Bouaoun et al., 2016), showed significantly elevated expression in cells with MDM2 degradation or knockdown in both p53-mutant and deleted TNBC cells (FDR<0.05; FIG. 28B; FIG. 29B). Notably, there was a highly significant correlation between p53 family-targeted genes with differential expression in MDA-MB-231 and MDA-MB-436 cells treated with YX-02-030 versus MDM2 knockdown (Spearman's p=0.873, P=0 for MDA-MB-231 and p=0.931, P=0 for MDA-MB-436; FIG. 28C; FIG. 29C), further illustrating the specificity of YX-02-030 for MDM2.

To independently validate the RNA-seq results, qRT-PCR was performed on a subset of the genes regulated by the p53 family that mediate apoptosis. Following MDM2 degradation or MDM2 knockdown in MDA-MB-231 and MDA-MB-436 cells, levels of apoptotic genes BAX, NOXA, PUMA, AEN, APAF1, TP53I3, and PIDD1 were significantly increased in both TNBC cell lines compared to controls (FIG. 28D; FIG. 29D). Thus, MDM2 loss upregulates genes transcriptionally regulated by the p53 family to induce apoptosis, but because p53 is inactivated, this indicated another family member is likely responsible.

MDM2 can bind and regulate p53 family members p73 (Balint et al., 1999; Dobbelstein et al., 1999; Zeng et al., 1999; Ongkeko et al., 1999) and p63 (Calabro et al., 2002; Kadakia et al., 2001), but the conditions in which this takes place, particularly in p53-inactivated cells remain unresolved. Because the transcriptionally active forms of p73 (TAp73) and p63 (TAp63) are capable of transactivating most of the same genes as p53 (Levine, 2020), we evaluated their expression. Upon MDM2-PROTAC treatment or MDM2 knockdown, levels of TAp73 protein increased in all five TNBC cell lines assessed (FIG. 28E, F; FIG. 30A-C); however, TAp63 was lowly expressed and its levels remained unchanged (FIG. 28E, F; FIG. 30A, B). Additionally, levels of TAp73 protein, but not TAp63, were increased in the xenograft tumors harvested 72 hours after PROTAC treatment and in the TNBC patient-derived explants following YX-02-030 treatment (FIG. 28G; FIG. 30C). In contrast, MDM2 inhibitor treatment did not increase TAp73 (FIG. 28E; 30A). Note that to effectively detect TAp73 and TAp63, proteins were isolated under conditions designed to extract transcription factors, which tend to be positively charged and/or chromatin-bound (FIG. 30D). Levels of ANp73, which lacks the N-terminal transactivation domain and can inhibit TAp73 ((Grob et al., 2001), in MDA-MB-231 and MDA-MB-436 cells were low and unaffected by MDM2 degradation or knockdown (FIG. 28E, F; FIG. 30A, B). Increased TAp73 protein was not due to increased transcription, as TAp73 mRNA levels were unaltered upon MDM2 loss (FIG. 30E). Both mRNA (FIG. 28D; FIG. 29D) and protein (FIG. 28E, F; FIG. 30A, B) levels of TAp73 apoptotic targets were upregulated with MDM2 loss in the TNBC cell lines, suggesting TAp73 was activated.

To further understand how MDM2 loss would activate TAp73, YX-02-030 binding was assessed in the p53/p73 binding pocket of MDM2 to determine if it would disrupt interactions with TAp73 in p53-inactivated TNBC cells, allowing TAp73 to move to chromatin to transcriptionally upregulate its apoptotic target genes. Immunoprecipitations of both MDM2 and TAp73 were performed in the presence of YX-02-030 or vehicle control in p53-mutant and deleted TNBC cells. The proteasome inhibitor MG132 was included to prevent proteasomal degradation of MDM2. In the YX-02-030-treated samples, there was markedly less TAp73 associated with MDM2, in both MDM2 and TAp73 immunoprecipitations (FIG. 28H; FIG. 30F), indicating the PROTAC was interfering with MDM2; TAp73 binding. Next, chromatin immunoprecipitation of TAp73 were performed after YX-02-030 treatment, and detected significant enrichment of TAp73 at the promoters of its apoptotic target genes (BAX, NOXA, PUMA, AEN, APAF1, PIDD1, TP53I3), but not at the promoter of AchR, which is not targeted by p53/TAp73 ((41), FIG. 28I; FIG. 30G). Taken together, PROTAC-mediated degradation of MDM2 prevents MDM2:TAp73 association in p53-inactivated TNBC cells, allowing activation of TAp73-mediated transcription of apoptotic genes.

To assess the requirements of p73 in mediating the effects of YX-02-030, TAp73 was knocked down in p53-mutant and deleted TNBC cells (FIG. 28J; FIG. 30H). These two shRNA were TAp73-specific, as they did not impact levels of mutant p53, TAp63, or ANp73. Although MDM2 protein was lost following YX-02-030 treatment, knockdown of TAp73 in both TNBC lines prevented upregulation of its apoptotic target genes (FIG. 28K; FIG. 30I), which was also reflected at the protein level (FIG. 28J; FIG. 30H). TAp73 knockdown also largely rescued the decrease in cell growth (FIG. 28L; FIG. 30J) and the cleavage of PARP (FIG. 28J; FIG. 30H), resulting from PROTAC-mediated MDM2 degradation. Collectively, these results indicate that YX-02-030 effectively targets MDM2 for degradation, activating TAp73-dependent apoptosis in p53-inactivated TNBC, and provides a future avenue for treatment of this deadly cancer.

### Example 3—MDM2 and VHL Binding

TABLE 3
MDM2 and VHL Binding Activity for Other Compounds

| | |
|---|---|
| MDM2-p53 | VHL-HIFa peptide |

IC₅₀, nM ± S.E.M.

| | | |
|---|---|---|
| Nutlin-3 | 89 ± 11 | >10000 |
| IK-01-186 | 2700 ± 330 | 1630 ± 169 |
| IK-01-189 | 32 ± 1.8 | 1560 ± 318 |
| JG-02-070 | >10000 | 1600 ± 168 |
| YX-02-023 | 11 ± 1.3 | >10000 |

### A. Inhibition of MDM2 Binding to p53 Using HTRF Technology

Both GST-MDM2 and HIS-p53 were expressed E Coli and purified by affinity chromatography. pGEX-4T MDM2 WT was a gift from Mien-Chie Hung (Addgene plasmid #16237; n2t.net/addgene:16237; RRID:Addgene_16237) and human p53-(1-393) was a gift from Cheryl Arrowsmith (Addgene plasmid #24859; http://n2t.net/addgene:24859; RRID:Addgene_24859). HTRF assays contained 1 nM GST-MDM2, 0.7 nM HIS-p53, 0.3 nM of anti-GST-Tb HTRF donor and 4 nM of anti-HIS-d2 HTRF acceptor antibodies (PerkinElmer) in 10 μL of 10 mM HEPES, pH 7.4, 100 mM NaCl, 5 mM MgCl2, 5 mM DTT, 0.01% Triton X-100 in white low-volume 384-well plates. Both GST-MDM2 and HIS-p53 were pre-incubated with their respective HTRF antibodies for 15 min. Test compounds were solubilized at 10 mM in DMSO and concentration series was added to the assay plate using the Echo 650 acoustic liquid handling system by direct dilution. The final amount of DMSO added to the assay was 100 nL (0.1%). Compounds were pre-incubated with 5 μL of GST-MDM2 for 15-30 min before 5 μL of HIS-p53/anti-HIS-d2 was added to the assay. After a 2 hr incubation, the HTRF signal was measured using the ClarioStar plate reader (BMG Labtech).

Data were then normalized to % inhibition, where 0% is the HTRF signal in the absence of compound, and 100% is the HTRF signal in the absence of GST-MDM2. IC₅₀ values were determined from nonlinear regression fits of a data to a four parameter dose-response equation using XLFit (IDBS). Data are the means±S.E.M. of three replications. The data is described in Table 3 above.

### B. Inhibition of HIF-1α Peptide Binding to the VHL Complex Using HTRF Technology

Assays contained 2 nM of HIS-VHL complex (R&D systems, E3-655-025), 2 nM of biotin-hydroxyproline-HIF1α peptide (Biotin-DLDLEMLAXYIPMDDDFQL (SEQ ID NO: 2), X=hydroxyproline), 0.3 nM anti-HIS-Tb HTRF donor antibody and 4 nM streptavidin-d2 HTRF acceptor (PerkinElmer) in a final volume of 10 μL of 25 mM HEPES, pH 7.4, 150 mM NaCl, 5 mM DTT, 0.005% Tween20 in white low volume 384-well plates. Both HIS-VHL complex and biotin-HIF1α peptide were pre-incubated with their respective HTRF antibodies for 15 min. Test compounds were solubilized at 10 mM in DMSO and concentration series was added to the assay plate using the Echo 650 acoustic liquid handling system by direct dilution. The final amount of DMSO added to the assay was 100 nL (0.1%). Compounds were pre-incubated with HIS-VHL complex before the addition of the biotin-HIF1α peptide. After a 2 hr incubation, the HTRF signal was measured using the ClarioStar plate reader (BMG Labtech). Data were then normalized to % inhibition, where 0% is the HTRF signal in the absence of compound, and 100% is the HTRF signal in the absence of HIS-VHL. IC50 values were determined from nonlinear regression fits of a data to a four parameter dose-response equation using XLFit (IDBS). Data are the means±S.E.M. of three replications. The data is described in Table 3 above.

### Example 4—Activity in Other Cell Lines

The PROTACs prepared herein were tested for their use in other cancer cell lines to determine activity in multiple different types of cancer. See the data presented in Table 4.

TABLE 4
MDM2-PROTAC Induced Cell Death of Other Cancer Cell Lines

| | | | YX-02- | YX-02- |
|---|---|---|---|---|
| | | | 030 | 228 |
| Cell line | Cancer type | Species | IC50 (μM) | IC50 (μM) |
| Raji | Burkitt lymphoma | Human | 2.8 | 5.9 |
| Ramos | Burkitt lymphoma | Human | 3.1 | 5.5 |
| SU-DHL-6 | Diffuse large B cell | Human | 3.6 | 2.9 |
| | lymphoma | | | |
| SU-DHL-8 | Diffuse large B cell | Human | 1.9 | 4.6 |
| | lymphoma | | | |
| HH | T cell lymphoma | Human | 6.3 | 5.5 |
| Hut-78 | T cell lymphoma | Human | 9.0 | 8.8 |
| SEM | B-ALL | Human | 3.2 | 4.2 |
| RS4; 11 | B-ALL | Human | 1.9 | 2.5 |
| MOLT1 | T-ALL | Human | 2.6 | 2.7 |
| SUP-T1 | T-ALL | Human | 5.7 | 6 |
| H1299 | Lung adenocarcinoma | Human | 4.2 | 5.3 |
| PC9 | Lung adenocarcinoma | Human | 4.5 | 4.6 |
| SKOV3 | Ovarian | Human | 5.7 | 3.2 |
| OVCAR3 | Ovarian | Human | 4.5 | ** |
| OVCAR4 | Ovarian | Human | 4.6 | 4.8 |
| KPCA | Ovarian | Mouse | 2.9 | 3.4 |
| Cal27 | Head & Neck | Human | 2.3 | ** |
| SCC-25 | Head & Neck | Human | 3.1 | ** |
| DE1069 | Sarcoma | Mouse | 6.1 | ** |

* Not that values may change up to ±1-2 μM depending on the cell density
** Not Tested

### REFERENCES

The following references, to the extent that they provide exemplary procedural or other details supplementary to those set forth herein, are specifically incorporated herein by reference:
Aziz et al., “Acquisition of p53 mutations in response to the non-genotoxic p53 activator Nutlin-3.” Oncogene 30, 4678-4686 (2011). (PMC3347888)
Balint et al., “Mdm2 binds p73 alpha without targeting degradation.” Oncogene 1999; 18(27):3923-9.
Bouaoun et al., “TP53 Variations in Human Cancers: New Lessons from the IARC TP53 Database and Genomics Data.” Hum Mutat 2016; 37(9):865-76.
Bouska & Eischen, “Murine double minute 2: p53-independent roads lead to genome instability or death.” Trends Biochem Sci. 2009 June; 34(6):279-86. Epub 2009 May 15. PMID: 19447627.
Burgess et al., “Clinical Overview of MDM2/X-Targeted Therapies.” Front Oncol 2016; 6:7.
Calabro et al., “The human MDM2 oncoprotein increases the transcriptional activity and the protein level of the p53 homolog p63.” J Biol Chem 2002; 277(4):2674-81.
Centenera et al., “A patient-derived explant (PDE) model of hormone-dependent cancer.” Mol Oncol 2018; 12(9):1608-22.
Chae et al., “Mitochondrial Akt Regulation of Hypoxic Tumor Reprogramming.” Cancer Cell 2016, 30, 257-272. PMID 27505672
Cox & Mann, “MaxQuant enables high peptide identification rates, individualized p.p.b.-range mass accuracies and proteome-wide protein quantification.” Nat Biotechnol. 2008, 26, 1367-1372 PMID 19029910
Ding et al., “Discovery of RG7388, a potent and selective p53-MDM2 inhibitor in clinical development.” J Med Chem 2013; 56(14):5979-83.
Dobbelstein et al., “Inactivation of the p53-homologue p73 by the mdm2-oncoprotein.” Oncogene 1999; 18(12):2101-6.
Donehower et al., “Mice deficient for p53 are developmentally normal but susceptible to spontaneous tumours.” Nature. 1992, 356, 215-21. PMID 1552940
Dotsch et al., “p63 and p73, the ancestors of p53.” Cold Spring Harb Perspect Biol 2, a004887 (2010). (PMC2926756)
Eischen, “Decreased Mdm2 levels after DNA damage: antibody masking or protein degradation?” Cell Cycle 2011; 10(9):1347.
Eischen & Lozano, “The Mdm network and its regulation of p53 activities: a rheostat of cancer risk.” Human Mutation 35(6):728-37, 2014 PMID 24488925
Feeley et al., “Mdm2 Is Required for Survival and Growth of p53-Deficient Cancer Cells.” Cancer Res 2017; 77(14):3823-33.
Frost et al., “Potent and selective chemical probe of hypoxic signalling downstream of HIF-alpha hydroxylation via VHL inhibition.” Nat Commun 2016; 7:13312.
Grier et al., “Conditional allele of mdm2 which encodes a p53 inhibitor.” Genesis. 2002, 32, 145-7. PMID 11857803
Grob et al., “Human delta Np73 regulates a dominant negative feedback loop for TAp73 and p53.” Cell Death Differ 2001; 8(12):1213-23
Harris & Levine, “The p53 pathway: positive and negative feedback loops” Oncogene 2005, 24, 2899-2908 PMID 15838523
Hunziker et al., “Stress-specific response of the p53-Mdm2 feedback loop.” BMC Syst Biol. 2010 Jul. 12; 4:94.
Jung et al., “TP53 mutations emerge with HDM2 inhibitor SAR405838 treatment in de-differentiated liposarcoma.” Nat Commun 7, 12609 (2016). (PMC5013668)
Kadakia et al., “Regulation of p63 function by Mdm2 and MdmX.” DNA Cell Biol 2001; 20(6):321-30.
Li et al., “Discovery of MD-224 as a First-in-Class, Highly Potent, and Efficacious Proteolysis Targeting Chimera Murine Double Minute 2 Degrader Capable of Achieving Complete and Durable Tumor Regression.” J Med Chem 2019; 62(2):448-66.
Liberzon et al., “The Molecular Signatures Database (MSigDB) hallmark gene set collection.” Cell Syst 2015; 1(6):417-25.
Levine, “p53: 800 million years of evolution and 40 years of discovery.” Nat Rev Cancer 2020; 20(8):471-80.
Melo & Eischen, “Protecting the genome from mdm2 and mdmx.” Genes Cancer. 2012 March; 3(3-4):283-90.
Michaelis et al., “Adaptation of cancer cells from different entities to the MDM2 inhibitor nutlin-3 results in the emergence of p53-mutated multi-drug-resistant cancer cells.” Cell Death Dis 2, e243 (2011). (PMC3252738)
Nalawansha & Crews, “PROTACs: An Emerging Therapeutic Modality in Precision Medicine.” Cell Chem Biol 2020; 27(8):998-1014.
Nag et al., “Targeting MDM2-p53 interaction for cancer therapy: are we there yet?” Curr Med Chem. 2014, 21, 553-74. PMID 24180275
Oliner et al., “The Role of MDM2 Amplification and Overexpression in Tumorigenesis.” Cold Spring Harb Perspect Med. 2016 Jun. 1; 6(6):a026336.
Olivier et al., “TP53 mutations in human cancers: origins, consequences, and clinical use.” Cold Spring Harb Perspect Biol 2, a001008 (2010). (PMC2827900)
Ongkeko et al., “MDM2 and MDMX bind and stabilize the p53-related protein p73.” Curr Biol 1999; 9(15):829-32.
Ray-Coquard et al., “Effect of the MDM2 antagonist RG7112 on the P53 pathway in patients with MDM2-amplified, well-differentiated or dedifferentiated liposarcoma: an exploratory proof-of-mechanism study.” Lancet Oncol 2012; 13(11):1133-40.
Rayburn et al., “MDM2 and human malignancies: expression, clinical pathology, prognostic markers, and implications for chemotherapy.” Curr Cancer Drug Targets 2005; 5(1):27-41.
Rew & Sun, “Discovery of a small molecule MDM2 inhibitor (AMG 232) for treating cancer.” Med Chem. 2014, 57, 6332-41. PMID 24967612
Riley & Lozano, “The Many Faces of MDM2 Binding Partners. Genes Cancer.” 2012 March; 3(3-4):226-39.
Schiewer et al., “Dual roles of PARP-1 promote cancer growth and progression.” Cancer Discov 2012; 2(12):1134-49.
Shin et al., “Structural convergence of unstructured p53 family transactivation domains in MDM2 recognition.” Cell Cycle 2015; 14(4):533-43. PMID 25591003
Skalniak et al., “Prolonged Idasanutlin (RG7388) Treatment Leads to the Generation of p5³-Mutated Cells.” Cancers (Basel). 2018 Oct. 24; 10(11):396. doi: 10.3390/cancers10110396. PMID: 30352966; PMCID: PMC6266412.
Soucy et al., “An inhibitor of NEDD8-activating enzyme as a new approach to treat cancer.” Nature 2009; 458(7239):732-6.
Vassilev et al., “In vivo activation of the p53 pathway by small-molecule antagonists of MDM2.” Science 2004; 303(5659):844-8.
Venkatanarayan et al., “Novel therapeutic interventions for p53-altered tumors through manipulation of its family members, p63 and p73.” Cell Cycle 15, 164-171 (2016). (PMC4825839)
Wu & Leng, “MDM2 mediates p73 ubiquitination: a new molecular mechanism for suppression of p73 function.” Oncotarget 2015; 6(25):21479-92.
Zeng et al., “MDM2 suppresses p73 function without promoting p73 degradation.” Mol Cell Biol 1999; 19(5):3257-66.

1. A compound of the formula: 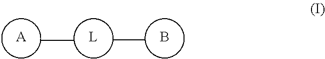
wherein:
A is a mdm2 inhibitor; wherein the mdm2 inhibitor comprises an imidazole core;
B is a ubiquitin ligase ligand; and
L is a linker group, wherein the linker group forms a covalent bond between the linker group and the mdm2 inhibitor and a covalent bond between the ubiquitin ligase ligand; wherein the linker group comprises at least one heteroatom selected from O, N, and S;
or a pharmaceutically acceptable salt thereof.

2. The compound of claim 1, wherein the mdm2 inhibitor is further defined by the formula: 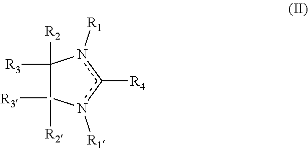
wherein:
R₁ and R₁′ are each independently a group of the formula: 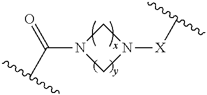
wherein:
X is -alkanediyl_{(C≤8)}—C(O)— or substituted -alkanediyl_{(C≤8)}—C(O)—; and
x or y are each independently 1, 2, or 3;
provided that either R₁ or R₁′ is absent when the nitrogen to which it is attached is a part of a double bond;
R₂ and R₂′ are each independently hydrogen or alkyl_{(C≤8)}, cycloalkyl_{(C≤8)}, alkenyl_{(C≤8)}, alkynyl_{(C≤8)}, aryl_{(C≤12)}, aralkyl_{(C≤12)}, or a substituted version of any of these groups;
R₃ and R₃′ are each independently alkyl_{(C≤8)}, cycloalkyl_{(C≤8)}, alkenyl_{(C≤8)}, alkynyl_{(C≤8)}, aryl_{(C≤12)}, heteroaryl_{(C≤12)}, aralkyl_{(C≤12)}, or a substituted version of any of these groups; and
R₄ is cycloalkyl_{(C≤8)}, aryl_{(C≤8)}, heteroaryl_{(C≤8)}, heterocycloalkyl_{(C≤8)}, or a substituted version thereof, wherein the group may be further substituted by one or more alkyl_{(C≤8)}, alkoxy_{(C≤8)}, alkylamino_{(C≤8)}, dialkylamino_{(C≤8)}, or a substituted version of these groups;
or a pharmaceutically acceptable salt thereof.

3. The compound of claim 2, wherein the mdm2 inhibitor is further defined: 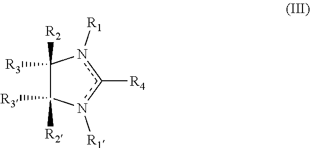
wherein:
R₁ and R₁′ are each independently a group of the formula: 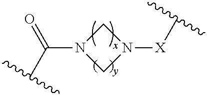
wherein:
X is -alkanediyl_{(C≤8)}—C(O)— or substituted -alkanediyl_{(C≤8)}—C(O)—; and
x or y are each independently 1, 2, or 3;
provided that either R₁ or R₁′ is absent when the nitrogen to which it is attached is a part of a double bond;
R₂ and R₂′ are each independently hydrogen or alkyl_{(C≤8)}, cycloalkyl_{(C≤8)}, alkenyl_{(C≤8)}, alkynyl_{(C≤8)}, aryl_{(C≤12)}, aralkyl_{(C≤12)}, or a substituted version of any of these groups;
R₃ and R₃′ are each independently alkyl_{(C≤8)}, cycloalkyl_{(C≤8)}, alkenyl_{(C≤8)}, alkynyl_{(C≤8)}, aryl_{(C≤12)}, heteroaryl_{(C≤12)}, aralkyl_{(C≤12)}, or a substituted version of any of these groups; and
R₄ is cycloalkyl_{(C≤8)}, aryl_{(C≤8)}, heteroaryl_{(C≤8)}, heterocycloalkyl_{(C≤8)}, or a substituted version thereof, wherein the group may be further substituted by one or more alkyl_{(C≤8)}, alkoxy_{(C≤8)}, alkylamino_{(C≤8)}, dialkylamino_{(C≤8)}, or a substituted version of these groups;
or a pharmaceutically acceptable salt thereof.

4. The compound of claim 2 further comprising: 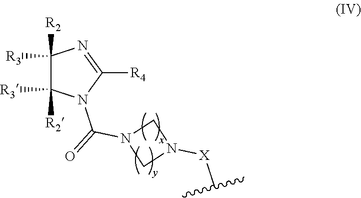
wherein:
R₂ and R₂′ are each independently hydrogen or alkyl_{(C≤8)}, cycloalkyl_{(C≤8)}, alkenyl_{(C≤8)}, alkynyl_{(C≤8)}, aryl_{(C≤12)}, aralkyl_{(C≤12)}, or a substituted version of any of these groups;
R₃ and R₃′ are each independently alkyl_{(C≤8)}, cycloalkyl_{(C≤8)}, alkenyl_{(C≤8)}, alkynyl_{(C≤8)}, aryl_{(C≤12)}, heteroaryl_{(C≤12)}, aralkyl_{(C≤12)}, or a substituted version of any of these groups; and
R₄ is cycloalkyl_{(C≤8)}, aryl_{(C≤8)}, heteroaryl_{(C≤8)}, heterocycloalkyl_{(C≤8)}, or a substituted version thereof, wherein the group may be further substituted by one or more alkyl_{(C≤8)}, alkoxy_{(C≤8)}, alkylamino_{(C≤8)}, dialkylamino_{(C≤8)}, or a substituted version of these groups;
or a pharmaceutically acceptable salt thereof.

5. The compound of claim 2, wherein R₁ is 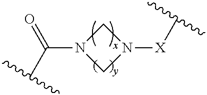
wherein:
X is -alkanediyl_{(C≤8)}—C(O)— or substituted -alkanediyl_{(C≤8)}—C(O)—; and
x or y are each independently 1, 2, or 3.

6. The compound of claim 5, wherein X is -alkanediyl_{(C≤8)}—C(O)—.

7-11. (canceled)

12. The compound of claim 2, wherein R₂ and/or R₂′ are each independently alkyl_{(C≤8)} or substituted alkyl_{(C≤8)}.

13-17. (canceled)

18. The compound of claim 2, wherein R₃ and/or R₃′ are each independently aryl_{(C≤12)} or substituted aryl_{(C≤12)}.

19-25. (canceled)

26. The compound of claim 2, wherein R₄ is aryl_{(C≤8)} or substituted aryl_{(C≤8)}, wherein the group is further substituted with one or more alkyl_{(C≤8)}, alkoxy_{(C≤8)}, alkylamino_{(C≤8)}, dialkylamino_{(C≤8)}, or a substituted version of these groups.

27-35. (canceled)

36. The compound of claim 2, wherein the mdm2 inhibitor is further defined as:

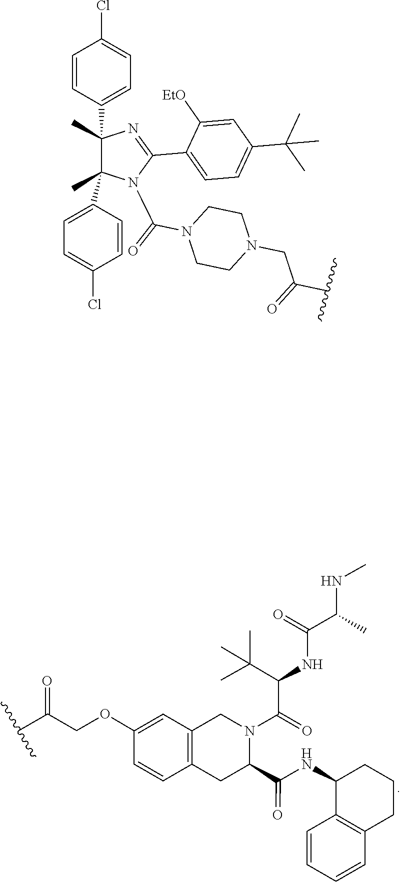 [SMILES: *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1 ; *C(=O)COc1ccc2c(c1)CN(C(=O)[C@H](NC(=O)[C@@H](C)NC)C(C)(C)C)[C@@H](C(=O)N[C@H]1CCCc3ccccc31)C2]

37. The compound of claim 1, wherein B is a Cereblon (CRBN) ligand, a Von-Hippel Lindau (VHL) ligand, a mouse double minute 2 homolog (MDM2) ligand, or a cellular inhibitor of apoptosis protein 1 (cIAP1) ligand.

38-46. (canceled)

47. The compound of claim 1, wherein L is a linker group of the formula: 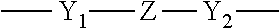
wherein:
Y₁ and Y₂ are each independently selected from —O—, —NRₐ—, —C(O)—, —C(O)O—, —OC(O)—, —C(O)NRₐ—, —NRₐC(O)—, —S(O)ₐ—, —S(O)ₐO—, —OS(O)ₐ—, —OS(O)ₐO—, wherein: Rₐ is hydrogen, alkyl(c>6), or substituted alkyl(c>6); and a is 0, 1, or 2; and
Z is alkanediyl_{(C≤12)}, one or more amino acid residues, or a group of the formula: —((Z₁)O)_{z}—, wherein: Z₁ is an alkanediyl_{(C1-4)} or a substituted alkanediyl_{(C1-4)} and z is 1-10 repeating units; or a combination thereof.

48-53. (canceled)

54. The compound according of claim 47, wherein Z is alkanediyl_{(C≤12)} or a substituted alkanediyl_{(C≤12)}.

55-56. (canceled)

57. The compound of claim 47, wherein Z is —((Z₁)O)_{z}—.

58-65. (canceled)

66. The compound of claim 1,
wherein the compound is further defined as: 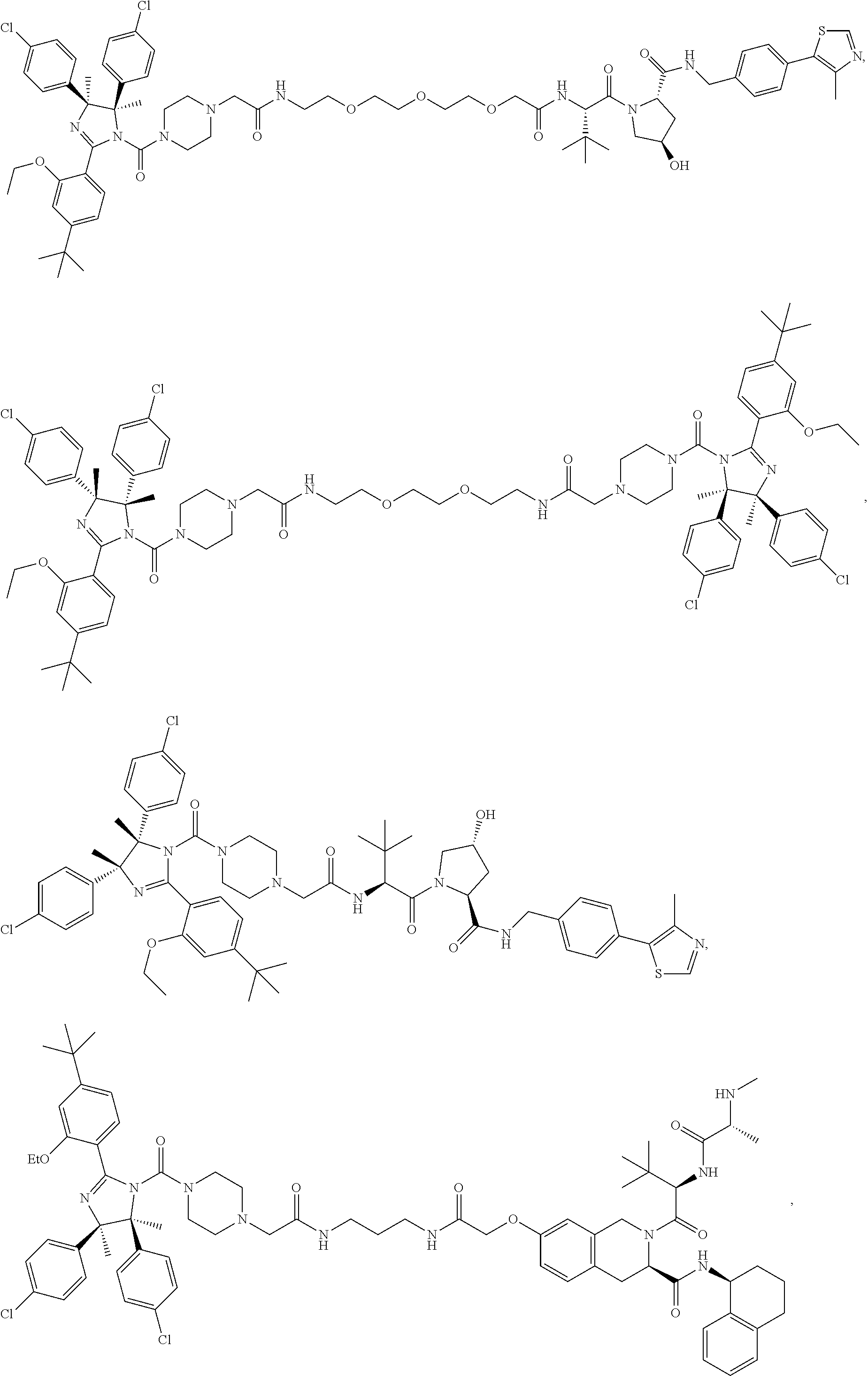
or a pharmaceutically acceptable salt thereof.

67. (canceled)

68. A pharmaceutical composition comprising:
(A) a compound of claim 1; and
(B) a pharmaceutically acceptable excipient.

69-70. (canceled)

71. A method of treating a disease or disorder in a patient in need thereof comprising administering to the patient in need thereof a therapeutically effective amount of a compound or of claim 1.

72-74. (canceled)

75. The method of claim 71, wherein the disease or disorder is a cancer comprises a mutated p53 gene or a deleted p53 gene.

76-95. (canceled)

96. A method of inhibiting cell replication by modulating mdm2 comprising contacting the cell with a compound of claim 1.

97. A method of modulating the activity of mdm2 in a cell comprising contacting the cell with a compound of claim 1.

98-100. (canceled)


SEQUENCE LISTING (2 sequences)

SEQ ID NO: 1 - protein, 12 aa, synthetic construct
NTNEPYTEGPTV

SEQ ID NO: 2 - protein, 19 aa, synthetic construct
DLDLEMLAXYIPMDDDFQL